(19) United States
(12) Patent Application Publication
(10) Pub. No.: US 2026/0314942 A1
(43) Pub. Date: Oct. 15, 2026

(54) LOCATION-BASED CARDIOVASCULAR MONITORING FOR PERFORMANCE AND MINDFULNESS SENSING

(71) Applicant: Tyler Joseph Monaldo, Odenton, MD (US)

(72) Inventor: Tyler Joseph Monaldo, Odenton, MD (US)

(21) Appl. No.: 19/177,536

(22) Filed: Apr. 12, 2025

(51) Int. Cl. A61B 5/0205; A61B 5/00; A61B 5/024; A61B 5/11; A61B 5/16; G16H 20/30

(52) U.S. Cl. CPC A61B 5/0205; A61B 5/1112; A61B 5/1118; A61B 5/165; A61B 5/742; G16H 20/30; A61B 5/024; A61B 2560/0242

(57) ABSTRACT

Location-based cardiovascular monitoring for performance and mindfulness sensing, in which a system receives, over time, user-specific biological data sensed by user-specific biological sensors, user-specific activity data sensed by user-specific activity sensors, user-specific environmental data sensed by user-specific environmental sensors, and user-specific location data sensed by user-specific location sensors. The system also receives, over time, location-specific environmental data sensed by location-specific environmental sensors. Based on the user-specific biological data, the user-specific activity data, the user-specific environmental data, the user-specific location data, and the location-specific environmental data, the system computes location-based user-specific performance scores, location-based user-specific mindfulness scores, location-specific performance scores, and location-specific mindfulness scores. The system performs at least one operation based on the computed scores.

### TECHNICAL FIELD

This disclosure relates to location-based cardiovascular monitoring for performance and mindfulness sensing.

### BACKGROUND

Sensors are known to measure characteristics of a performance or mindfulness facility. For example, temperature sensors are known to measure an indoor temperature within a facility. Air quality sensors are also known to measure a quality of the air within a facility. Other types of sensors may be used to measure other characteristics of a facility, such as water temperature and chemical levels of a swimming pool within the facility.

Sensors also are known to sense characteristics of a person's cardiovascular health and performance. For instance, sensors can track health-related characteristics of a person, including heart rate and respiratory-related metrics. Sensors also are known to measure athletic performance, such as time spent exercising and rates at which the person is completing activities. Sensors further are known to measure attributes of mindfulness, such as sensors to measure movement, heart rate, and brain activity during mindfulness activities.

Although facility-related and user-related sensors are known, the two types of sensing techniques are not sufficiently correlated to enable an analysis of a facility's impact on cardiovascular health, performance of athletes competing or practicing at the facility, and/or mindfulness states of people at the facility or location. In some examples, the present disclosure improves on prior sensing techniques by using location data to objectively characterize a facility's impact on health, performance, and/or mindfulness.

### SUMMARY

Techniques are described for location-based cardiovascular monitoring for performance and mindfulness sensing. For example, an electronic system configured to perform location-based cardiovascular monitoring for performance and mindfulness sensing includes a plurality of user devices and a plurality of location-specific devices. The plurality of user devices each include or are connected to (1) at least one user-specific biological sensor configured to sense biological characteristics of a specific user, (2) at least one user-specific activity sensor configured to sense activity characteristics of the specific user, (3) at least one user-specific environmental sensor configured to sense environmental characteristics of an area where the specific user is located when the biological and activity characteristics of the specific user are sensed, and (4) at least one user-specific location sensor configured to sense a location of the specific user when the biological and activity characteristics of the specific user are sensed. The plurality of location-specific devices each include or are connected to at least one location-specific environmental sensor configured to sense environmental characteristics at a specific location.

The electronic system also includes at least one database configured to store (1) user-specific biological data sensed by user-specific biological sensors, (2) user-specific activity data sensed by user-specific activity sensors, (3) user-specific environmental data sensed by user-specific environmental sensors, (4) user-specific location data sensed by user-specific location sensors, and (5) location-specific environmental data sensed by location-specific environmental sensors. The electronic system further includes at least one monitoring server configured to communicate with the plurality of user devices, the plurality of location-based devices, and the at least one database.

The at least one monitoring server includes one or more processors and one or more computer-readable media storing instructions that, when executed, cause at least one of the one or more processors to perform operations. The operations includes receiving, over time and from the plurality of user devices, (1) user-specific biological data sensed by user-specific biological sensors configured to sense biological characteristics of specific users, (2) user-specific activity data sensed by user-specific activity sensors configured to sense activity characteristics of specific users, (3) user-specific environmental data sensed by user-specific environmental sensors configured to sense environmental characteristics of areas where specific users are located when biological and activity characteristics are sensed, and (4) user-specific location data sensed by user-specific location sensors configured to sense locations of specific users when biological and activity characteristics are sensed. The operations also include receiving, over time and from the plurality of location-specific devices, location-specific environmental data sensed by location-specific environmental sensors configured to sense environmental characteristics at specific locations and storing, in the at least one database, the received (1) user-specific biological data, (2) user-specific activity data, (3) user-specific environmental data, (4) user-specific location data, and (5) location-specific environmental data in association with timing data indicating a time of sensing.

In addition, the operations include, based on the stored (1) user-specific biological data, (2) user-specific activity data, (3) user-specific environmental data, (4) user-specific location data, and (5) location-specific environmental data, computing location-based user-specific performance scores that each measure health and performance levels for a specific user at a specific location, computing location-based user-specific mindfulness scores that each measure mindfulness levels for a specific user at a specific location, computing location-specific performance scores that each measure health and performance levels associated with a specific location, and computing location-specific mindfulness scores that each measure mindfulness levels associated with a specific location. The operations also include performing at least one operation based on the computed location-based user-specific performance scores, the computed location-based user-specific mindfulness scores, the computed location-specific performance scores, and the computed location-specific mindfulness scores.

Implementations of the described techniques may include hardware, a method or process implemented at least partially in hardware, or a computer-readable storage medium encoded with executable instructions that, when executed by a processor, perform operations.

The details of one or more implementations are set forth in the accompanying drawings and the description below. Other features will be apparent from the description and drawings, and from the claims.

### DESCRIPTION OF DRAWINGS

FIG. 1 illustrates an example of performing location-based cardiovascular monitoring for performance and mindfulness sensing.

FIG. 2 illustrates an example system.

FIGS. 3-5, 7, 8, 10, 12, and 14 are flow charts of example processes.

FIGS. 6, 9, 11, 13, and 15 illustrate example user interfaces.

### DETAILED DESCRIPTION

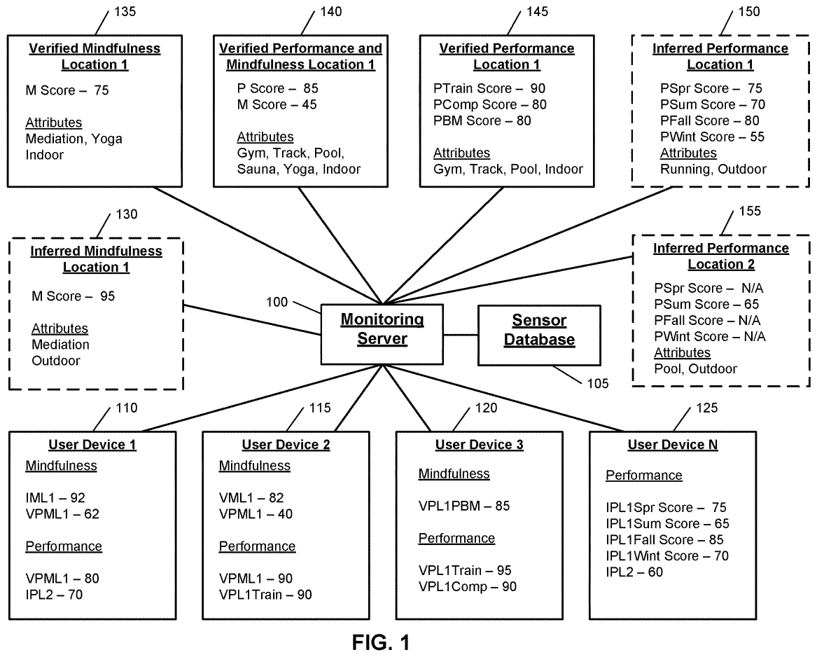

FIG. 1 illustrates an example of location-based cardiovascular monitoring for performance and mindfulness sensing. As shown in FIG. 1, the system includes a monitoring server 100 that communicates with a sensor database 105 and multiple user devices 110, 115, 120, 125. Although four user devices are shown, the system may include many more user devices.

The monitoring server 100 receives user-specific sensor data from each of the user devices and stores it in the sensor database 105. The user-specific sensor data may include biological data that measures biological aspects of a user. The biological data may include cardiovascular data, neurological data, analyte data, and any other type of bio-sensor data that is useful in measuring performance and/or mindfulness. The cardiovascular data may include data related to the heart, such as heart rate or heart rate variability. The cardiovascular data also may include data related to blood pressure and data related to respiratory function, such as blood oxygen level or breathing rate. The cardiovascular data may be sensed using any type of sensor configured to sense heart activity, blood pressure, or respiratory function. The sensors may include PPG sensors, ECG sensors, SPO2 monitors, pulse oximeters, etc.

The neurological data includes data from neurological sensors that monitor and analyze neural activity in the brain or muscles. The neurological sensors may include wireless brain sensors, implantable neural sensors, wearable muscle-centric sensors, neural dust, or any other types of sensors that are capable of sensing brain activity in a manner that generates data relevant to a person's mindfulness.

The analyte data includes data from bio-sensors configured to sense analytes in a person's body. The analyte sensors may include glucose monitors, sweat sensors, electrochemical sensors, optical bio-sensors, piezoelectric bio-sensors, thermal bio-sensors, or any other types of sensors that are able to sense analytes in a person's body.

The user-specific sensors also include activity sensors that sense a user's activity level. The activity sensors, particularly in the context of monitoring movement, health, and behavior, include accelerometers, gyroscopes, and magnetometers, often integrated into wearable devices like fitness trackers and smartwatches. The accelerometers measure acceleration in various directions (x, y, z axes), providing information about changes in speed and direction of movement. Accelerometers may be used for detecting steps, walking, running, and other activities involving changes in motion. Gyroscopes detect angular velocity and rotation, providing insight into how the body is turning or rotating in space. Gyroscopes may be used for tracking body orientation and movements like twisting, turning, and tilting and may be used to detect performance (e.g., weight lifting) and/or mindfulness (e.g., yoga) activities. Magnetometers measure the Earth's magnetic field and can be used to determine direction and orientation. Magnetometers provide context for movement and can be used in conjunction with other sensors for more comprehensive activity recognition. The activity sensors also may include inertial measurement units (IMUs). IMUs combine accelerometers, gyroscopes, and sometimes magnetometers to provide a comprehensive set of motion data. IMUs may be included in wearable devices for tracking a wide range of human activities.

The user-specific sensors also include location sensors that are configured to track a location of a user device. The location sensors may include GPS sensors, beacon-based location sensors, triangulation-based location sensors, or any other types of sensors that can determine a user's location and report it to the server 100.

The user-specific sensors further include environmental sensors that are configured to sense environmental conditions at a location of a user. The environmental sensors may include noise sensors, light sensors, air temperature sensors, water temperature sensors, air quality sensors, wind speed sensors, water quality sensors, such as sensors that sense chlorine levels in water, or any other types of environmental sensors that sense environmental conditions relevant to a user's performance or mindfulness.

The user devices may be smart phones that collect user-specific sensor data from sensors associated with the smart phone. The user devices also may be wearable devices, such as fitness trackers, smartwatches, and other wearable devices are equipped with various sensors to track activity, heart rate, sleep patterns, and other health metrics.

The monitoring server 100 continuously receives sensor data from the user devices 110, 115, 120, 125 and stores the received sensor data in the sensor database 105. Over time, the sensor database 105 establishes a database of sensor data that aggregates and tracks biological data, activity data, location data, and environmental data at different times.

The monitoring server 100 analyzes the data stored in the sensor database 105 to determine user-specific and location-specific performance and mindfulness scores. For example, the monitoring server 100 clusters data into user-specific and location-specific clusters of data such that the monitoring server 100 can assess performance and/or mindfulness at specific locations for specific users. In this example, within each cluster, the monitoring server 100 analyzes the biological data, the activity data, and/or the environmental data to identify performance and/or mindfulness events. For each identified event, the monitoring server 100 analyzes the biological data, the activity data, and the environmental data sensed at a time of the event to determine a performance and/or mindfulness score. In this regard, the monitoring server 100 may use any of the techniques described throughout this disclosure to generate the performance and/or mindfulness scores. In general, the monitoring server 100 uses a process that weights different biological parameters in relation to activity data and environmental data to make an assessment of the user's performance and/or mindfulness at the specific location for the identified events. For instance, the monitoring server 100 may use different threshold ranges for biological data (e.g., heart rate, HRV, blood oxygen, etc.) for different activity levels (e.g., intense running, walking, swimming, yoga, etc.) at different environmental conditions (e.g., different temperatures, different wind speeds, etc.) and compute a performance and/or mindfulness score based on how the biological data compares to the activity level and environmental conditions associated with a performance or mindfulness event.

In some implementations, the monitoring server 100 may use machine learning to determine performance and/or mindfulness scores. In these implementations, the monitoring server 100 may input the sensor data from sensor database 105 into a machine learning model that assesses the data. The machine learning process analyzes the sensor data, detects patterns within it, and uses the detected patterns to identify events and assess performance and mindfulness levels for each of the identified events. The monitoring server 100 may use any type of machine learning, such as neural networks or large language models, to analyze the sensor data.

The monitoring server 100 also detects locations where performance and mindfulness activities occur. By processing the sensor data from sensor database 105, the monitoring server 100 may identify locations where users typically engage in performance and/or mindfulness activities. The monitoring server 100 may compare identified locations with known location data that identifies locations known to be places where performance and/or mindfulness activities occur. Based on the comparison, the monitoring server 100 may verify certain locations that match in terms of types of activities detected, and the types of activities that would be expected from the known location data.

The monitoring server 100 also may infer locations that do not match the known location data. These locations may be outdoor spaces where people engage in performance and/or mindfulness activities, but are not associated with a facility that is described as being used for performance and/or mindfulness activities in the known location data. By inferring locations, the monitoring server 100 may improve the processing of location data because the monitoring server 100 is able to assess performance at locations that would otherwise not be considered for performance and/or mindfulness activities. The monitoring server 100 also can update the known location data to more quickly identify locations where users are enjoying performance and/or mindfulness activities. Because the monitoring server 100 automatically identifies and detects the inferred locations, the processing of the system is improved and does not require a user to have to provide input to identify inferred locations where performance and/or mindfulness activities occur.

In the example shown in FIG. 1, the monitoring server 100 has detected six locations 130, 135, 140, 145, 150, 155. Although six locations are shown, the monitoring server 100 may detect and analyze many more locations. In the example shown in FIG. 1, three locations have been verified and three locations have been inferred.

Location 130 is an inferred mindfulness location. The monitoring server 100 detected the inferred mindfulness location 130 as an outdoor space where meditation frequently occurs. The monitoring server 100 detected the inferred mindfulness location 130 by clustering a significant amount of biological, activity, and/or environmental data that suggested meditation activities frequently occur at location 130. The monitoring server 100 detected the meditation activities using user-specific sensor data that was aggregated and correlated to the location 130. For example, the monitoring server 100 detected a relatively large number of people that would travel to location 130 and spend time with a relatively low activity level. The monitoring server 100 also may consider the pose of the users while maintaining a low activity level, such as a seated pose that indicates potential meditation. The monitoring server 100 also considers biological data and detected that users with a low level of activity experienced biological sensing that suggested meditation, such as low heart rate, low respiratory rate, and/or neurological data that suggested brain activity consistent with meditation. The monitoring server 100 analyzes the activity data with the biological data and detected a large number of meditation events occurring at the location 130 and therefore inferred the location 130 as a mindfulness location where meditation occurs.

The monitoring server 130 also detected the location 130 as an outdoor space based on environmental data collected by user sensors, such as temperature and wind speed that aligns with outdoor spaces. The monitoring server 100 further may consider weather data and whether the user-specific environmental data sensed is consistent with expected outdoor weather at the location 130. In this way, the monitoring server 100 improves the processes for monitoring user performance, health, and wellness by inferring the existence of location 130 as a mindfulness location and also inferring attributes of the location that are helpful in further processing additional data sensed at the location 130.

The monitoring server 100 also determines a mindfulness score for the location 130 as 95 out of 100. The monitoring server 100 determines the mindfulness score by evaluating the user-specific biological data, activity data, and/or environmental data sensed at the location 130 during the meditation activities. The monitoring server 100 compares the biological data, activity data, and/or environmental data to data that is preferred for high-quality meditation. Based on this comparison, the monitoring server 100 determined that the meditation activities occurring at the location 130 are of high-quality and computed a score of 95 to represent the collective quality of the meditation activities occurring at the location 130. The monitoring server 100 may analyze the activity data to confirm that users are able to sit still and comfortably during meditation at the location 130. The monitoring server 100 also may analyze the biological data to determine whether biological attributes suggest high-quality meditation, such as low heart rate, low respiratory rate, and/or neurological activity that indicates high-quality meditation. The monitoring server 100 may assess the data in association with a variety of rules and/or thresholds to weight the different characteristics and determine the mindfulness score for the location 130, considering and accounting for data from all users that have user-specific data sensed at the location 130. The monitoring server 100 also may input the data collected at location 130 into a machine learning process that evaluates the quality of meditation activities occurring at the location 130. The monitoring server 100 also may consider environmental data and determine whether the environmental characteristics at the location 130 are characteristics that are suitable for meditation activities, such as low wind speed, and pleasant temperatures. In analyzing all of the user-specific data for all users at the location 130, the monitoring server 100 is able to develop a mindfulness score that reflects the quality of mindfulness activities that occur at inferred location 130.

Location 135 is a verified mindfulness location. The monitoring server 100 determined that location 135 is a verified mindfulness location by comparing the sensor data sensed at the location 135 with known location data (e.g., map data or a directory of businesses with corresponding addresses) that indicates that location 135 is the site of a yoga studio where users participate in mindfulness activities. The monitoring server 100 may verify that the user-specific sensor data sensed at location 135 is consistent with mindfulness activities, such as meditation and yoga. The monitoring server 100 also may assess the user-specific attributes to confirm that the environmental data is consistent with data at an indoor yoga studio. When the user-specific sensor data is consistent with the activities expected at an indoor yoga studio, the monitoring server 100 verifies the location 135 as a verified mindfulness location, thereby validating the data in the known location data and improving the processing of sensor data at different locations by verifying that the known location data (e.g., map data or a directory of businesses with corresponding addresses) is accurate or not.

The monitor server 100 generates a mindfulness score at the location 135 and determined that location 135 has a score of 75 out of 100. The monitoring server 100 computes the mindfulness score using techniques similar to those described above for location 130. In this case, the monitoring server 100 considers both meditation events and yoga events to generate an overall mindfulness score that reflects the quality of the meditation and yoga activities that occur at location 135. In computing a score of 75, the monitoring server 100 determined that the mindfulness activities at location 135 were of lower quality than those at location 130, but still at an above average quality for mindfulness activities. In some implementations, the monitoring server 100 may separate the meditation and yoga activities into separate buckets of sensor data and generate a meditation-specific mindfulness score for the location 135 and a yoga-specific mindfulness score for the location 135.

Location 140 is a verified performance and mindfulness location. The monitoring server 100 verified location 140 as both a performance and mindfulness location by comparing the activities that occur at location 140 to those that would have been expected from a facility with the attributes from known location data indicating that a facility for performance and mindfulness exists at location 140. For instance, the location 140 may correspond to a fitness facility that offers yoga classes and sauna services. To verify location 140, the monitoring server 100 detected gym activities, such as weightlifting, track activities, such as running, and pool activities, such as swimming, to determine that the performance activities occurring at the location 140 are consistent with those that are expected at a large fitness center with a gym, track, and pool. The monitoring server 100 also identified yoga activities occurring at the location 140 based on activity data indicating groups of users performing yoga activities at the location 140 at the same time. The monitoring server 100 also verified presence of a sauna based on activity data suggesting that users were sitting in the sauna as well as biological data indicating user presence in a sauna, such as sweat sensing and heart rate sensing, and environmental data suggesting presence in the sauna, such as a high environmental temperature.

Because the monitoring server 100 verified the location 140 as both a performance and mindfulness location, the monitoring server 140 computes both a performance score and a mindfulness score for the location 140. In some implementations, the monitoring server 100 separates mindfulness data associated with detected mindfulness activities, performance data associated with detected performance activities, and miscellaneous data that has not been identified as corresponding to a mindfulness or performance activity (e.g., sensed data for pre-or post-workout or yoga as users enter and exit the facility) into separate buckets of sensor data. In these implementations, the monitoring server 100 may discard the miscellaneous data, use the mindfulness data to generate a mindfulness score for the location 140 that accounts for all types of mindfulness activities occurring at the location 140, and use the performance data to generate a performance score for the location 140 that accounts for all types of performance activities occurring at the location 140 (e.g., weightlifting, running, and swimming). In addition to generating overall scores, the monitoring server 100 may generate activity specific scores, such as pairing the overall performance score that accounts for weightlifting, running, and swimming activities with a weightlifting performance score, a running performance score, and a swimming performance score to enable users to perceive how different activities are influencing the overall performance score and to determine whether the location 140 is good for certain activity types while being subpar for others.

The monitoring server 100 may compute the performance and mindfulness scores for the location 140 using any of the techniques described throughout this disclosure. In this example, the monitoring server 100 compute a performance score of 85 out of 100 for location 140 and a mindfulness score of 45 out of 100 for the location 140. The scores indicate to users that the location 140 has above average performance characteristics, but below average mindfulness characteristics, suggesting that users may want to consider location 140 for performance activities, but consider other locations for mindfulness activities.

Location 145 is a verified performance location. The monitoring server 100 verified the location 145 as a performance location using techniques similar to those discussed above for verifying location 140 as a verified performance location. In this example, the monitoring server 100 determined that the environmental sensor data at location 145 indicates characteristics consistent with an indoor athletic facility. The monitoring server 100 also detected activities that are consistent with a gym, track, and pool, thereby confirming the attributes available at location 145 are consistent with those that are expected from the known location data.

For location 145, the monitoring server 100 detected that both training activities and competition activities occur at location 145. For example, the monitoring server 100 determines that the location 145 is a location where swim groups train at swim practice during certain times, but that at other times the location 145 is used as a venue for swimming competitions. The monitoring server 100 is able to distinguish between training and competition activities by analyzing the user specific sensor data. For instance, when a large number of users are engaging in swimming activities at a relatively continuous pace for an extended period of time, the monitoring server 100 detects users participating in training activities during a swim practice. These types of training activities contrast with competition swimming in which swimmers participate in a relatively short races spaced apart by a time for rest. When the monitoring server 100 detects a significant number of users that are using the facility at location 145 in a manner consistent with competition swimming (e.g., a few relatively short swim events spaced with breaks of 30 to 90 minutes), the monitoring server 100 detects a competition swimming event.

The monitoring server 100 also may consider the intensity of the user specific activity and biological sensors during the training and competition events to confirm whether specific events are associated with training or competition. For instance, competition events are typically associated with activity and biological data that indicates short sequences of high intensity activity. In contrast, training activities are associated with activity and biological data that indicates more continuous swimming events at a lower intensity level. By looking at patterns of swim activities and the types of swimming occurring during those activities, the monitoring server 100 is able to detect competition versus training activities. The monitoring server 100 further may use machine learning processes to identify training patterns of events versus competition patterns of events to distinguish between training and competition activities.

The monitoring server 100 also may use scheduling information to verify the training and competition activities detected, such as by evaluating a swim practice schedule and/or a swim meet schedule to verify that training activities were detected during the swim practice activities or detected during the scheduled swim meets. By segmenting data into training and competition data, the monitoring server 100 is able to provide better performance scoring that is targeted to both training and competition. For instance, a particular location may be great for training, but less preferred for competition. In contrast, a different location may be great for competition, but subpar for training. By segmenting data into competition and training data, the monitoring server 100 is able to compute better, more granular information and therefore improve processes for automatically assessing performance at a location.

In the example shown in FIG. 1, the monitoring server 100 segments training data and competition data into separate buckets. The monitoring server 100 then processes the training data to determine a performance based training score that reflects the quality of training activities that occur at location 145. The monitoring server 100 also processes the competition data to determine a performance based competition score that reflects the quality of competition activities that occur at location 145. In this example, the monitoring server 100 determines that location 145 has a training score of 90 out of 100 and a competition score of 80 out of 100, suggesting that, while the location 145 is above average for both training and competition, it is slightly better as a training venue than a competition venue. In some examples, the monitoring server 100 may use different processes for evaluating training versus competition data. Because competition events are shorter and have higher intensity, the monitoring server 100 may weight sensor data differently for competition events versus training events.

In some examples, the monitor server 100 also considers environmental data in assessing the training and competition scores for the location 145. In these examples, the monitoring server 100 assesses the environmental characteristics during each type of event and determines whether there are any differences that can be used to help the location 145 improve either of its training or competition activities. For example, the monitoring server 100 may detect that environmental conditions are worse during competition events, potentially due to the presence of spectators. In this regard, the monitoring server 100 may determine that the presence of spectators increases the temperature in the facility slightly as compared to the temperature in the facility during training activities. The monitoring server 100 may detect that the increase in temperature correlates to a decrease in performance and can suggest that the facility lower its temperature before competition events, such that the temperature increase due to spectators will align the facility's temperature more closely to the temperature that is typical for training activities. The monitoring server 100 may consider air temperature, water temperature, chemical levels in a pool, and/or air quality sensing in evaluating the environmental characteristics during both training and competition activities at the location 145.

Because the monitoring server 100 detected competition activities occurring at location 145, the monitoring server 100 also computed a performance based mindfulness (PBM) score for the location 145. The performance based mindfulness score reflects mindfulness of competitors during competition events detected at the location 145. To determine the performance based mindfulness score, the monitoring server 100 evaluates mindfulness characteristics for competitors prior to an event, during an event, and/or after a competition event to assess whether a competitor's mindfulness is impacting the competitor's performance. For instance, many competitors suffer from performance anxiety in that anxiety impacts the competitors ability to perform at a high level during the competition event. Because the monitoring server 100 is able to detect when competition events occur, the monitoring over 100 is able to assess mindfulness characteristics of competitors prior to the competition events. For example, the monitoring server 100 is able to evaluate user specific sensor data and determine whether the user specific sensor data suggests a good state of mind heading into a competition. In this example, the monitoring server 100 may consider activity sensor data to evaluate a user's activity level prior to a competition. In this regard, the monitoring server 100 determines whether the competitors activity level is low and calm before a competition event or, in contrast, high and fidgety in a manner that suggests that the competitor is nervous. The monitoring server 100 also considers biological data to assess the users mindfulness prior to a competition event. For the biological data, the monitoring server 100 may consider heart rate, temperature, respiratory rate, and/or neurological data to evaluate whether the competitor is calm before competition or experiencing anxiety. In this regard, the monitoring server 100 may detect anxiety when the competitor has a relatively high heart rate, a relatively high temperature, and a relatively high respiratory rate.

The monitoring server 100 considers all user specific sensor data to compute an overall performance based mindfulness score for the location. Monitoring server 100 may consider mindfulness before, during, and after competition events and determine a score that reflects the mindfulness of competitors at the specific location. The monitoring server 100 may be able to sense mindfulness characteristics at competition events, relative to training events and relative to other locations. By considering relative mindfulness states during different types of activities, such as training versus competition, and at different locations, the monitoring server 100 can generate a performance based mindfulness score that reflects the performance based mindfulness at location 145. In this example, the monitoring server 100 determines that performance based mindfulness at the location 145 is relatively high and generated a score of 80 out of 100. By providing a performance based mindfulness score, the monitoring server 100 is able to analyze and assess whether the mindfulness characteristics of the location 145 is contributing to decreased performance at location 145. This additional information may be useful to users and may help diagnose instances of performance anxiety or other types of conditions where a user's mindfulness state impacts performance during a competition. Further, the monitoring server 100 may assess performance based mindfulness across different locations and different types of users to identify attributes of locations and users that are best suited for competitions where mindfulness does not impact performance. In this regard, the monitoring server 100 may be able to help users identify locations that are better suited for competition performance events and/or help locations adjust mindfulness characteristics to improve the mindfulness of the competitors competing at the performance location.

Location 150 is an inferred performance location. As shown in FIG. 1, the monitoring server 100 detected performance activities occurring at location 150 and determined that location 150 is an outdoor space where running often occurs. The outdoor space may be an outdoor track or path that is not necessarily documented within known location data. The monitoring server 100 is able to infer location 150 as an outdoor running space based on the types of performance activities occurring at location 150 and the environmental characteristics indicating that the location is outdoors.

The monitoring server 100 also determines that location 150 is a seasonal location where performance activities differ across different seasons. For instance, monitoring server 100 may detect running activities at a higher volume during seasons where the weather is nicer and at a lower volume during seasons where the weather is worse. The monitoring server 100 also may detect the location 150 as a seasonal location by evaluating and determining that performance at the location 150 differs based on the season. For example, performance for outdoor running may be better during more mild seasons, such as spring or fall compared to seasons where the weather is harsher, such as summer when it is hot and winter when it is cold.

Because the monitoring server 100 detected the location 150 as a seasonal location, the monitoring server 100 determined seasonal performance scores for the location 150. For instance, the monitoring server 100 bucketed the sensor data into buckets corresponding to spring, summer, fall, and winter. The monitoring server 100 then evaluated the spring data to determine a spring performance score of 75 out of 100, evaluated the summer data to determine a summer performance score of 70 out of 100, evaluated the fall data to determine a fall performance score of 80 out of 100, and evaluated the winter data to determine a winter performance score of 55 out of 100. With the scores, the monitoring server 100 was able to detect that performance at the location 150 dipped most significantly in the winter where the temperatures are cold. The monitoring server 100 may use any of the techniques described throughout this disclosure to generate the seasonal scores for the location 150.

Location 155 is an inferred performance location. In this instance, the monitoring server 100 also detected the location 155 as a seasonal location where performance events only occur at a specific time of year. In this case, the monitoring server 100 detected swimming events occurring at location 155, but only in the summer. Based on detecting swimming events occurring at location 155 only during summer months, the monitoring server 100 was able to infer that location 155 corresponds to an outdoor pool. As shown in FIG. 1, the monitoring server 100 only generates a summer specific performance score for the location 155 and lists the spring, fall, and winter scores as being not available. To generate the summer performance score, the monitoring server 100 evaluates the swimming events that occurred during the summer months at location 155 to compute a score of 65 out of 100 using the techniques described throughout this disclosure.

In some implementations, the monitoring server 100 also receives environmental data from devices at various performance and mindfulness locations. In these implementations, the monitoring server 100 factors the environmental data from the location-based sensors into the analysis of the user-specific sensor data. The monitoring server 100 may compare the location-based environmental data to the user-specific environmental data to verify accuracy of environmental sensing. In this way, the monitoring server 100 is able to determine whether a user's environmental data is accurate or not. The monitoring server 100 also may determine that a location's sensors are inaccurately sensing environmental data when user-specific environmental data sensed at the location consistently senses environmental data that is different than the environmental data sensed by the location's sensors. In either scenario, the monitoring server 100 is able to alert the location or the user of the discrepancy in environmental sensing, which may cause the user or the location to improve its environmental data sensing, for example, by replacing its environmental sensors. Because the monitoring server 100 considers data from many locations and many user devices, the monitoring server 100 may process data to more quickly identify malfunctioning sensors to improve sensing performance at locations and user devices. The monitoring server 100 also may exclude environmental data that has been determined to have been inaccurately sensed, thereby improving the assessment of performance and mindfulness at the locations.

Each of the user devices 110, 115, 120, and 125 displays a user interface that presents user-specific mindfulness and/or performance scores. The mindfulness and performance scores may be computed using any of the techniques described throughout this disclosure with the data limited to the specific user associated with each device. The user device 110 is associated with a first user and presents mindfulness and performance scores computed for the first user. As shown in FIG. 1, the first user has computed mindfulness scores for the inferred mindfulness location 1 and the verified performance and mindfulness location 1. The scores were computed for these locations based on the user's participation in mindfulness activities at the two locations. As shown in FIG. 1, the first user has a relatively high mindfulness score at inferred mindfulness location 1, but an average score for mindfulness activities at verified performance and mindfulness location 1. From the scores, the first user can determine that the first user achieves better mindfulness results when engaging in mindfulness activities at inferred mindfulness location 1. The first user also has performance scores at verified performance and mindfulness location 1 and inferred performance location 2. As shown, the first user was given a score of 80 at verified performance and mindfulness location 1, suggesting that the user's performance at verified performance and mindfulness location 1 is better for performance activities than for mindfulness activities at the same location. The first user also has a score of 70 for performance activities at inferred performance location 2.

The user device 115 is associated with a second user and presents mindfulness and performance score specific to the second user. Similar to the first user, the second user has computed mindfulness scores at two locations. In this example, the second user has a computed mindfulness score of 82 at verified mindfulness location 1 and a computed mindfulness score of 40 at verified performance and mindfulness location 1. From the scores, the second user can determine that the second user has better mindfulness results when engaging in mindfulness activities at verified mindfulness location 1 as compared to verified performance and mindfulness location 1. The second user also has computed performance scores for verified performance and mindfulness location 1 as well as verified performance location 1. Each of the scores was computed to be 90, suggesting that the second user has relatively high performance at each of these locations. The performance score at verified performance location one is indicated as a training performance score because the second user has only engaged in training activities at verified performance location 1. Specifically, the monitoring server 100 has gathered data based on the second user's performance activities when located at verified performance location 1 and determined that the second user engages in training activities, but not competition activities. Based on detecting that the second user engages in training activities at verified performance location 1, the monitoring server 100 computes and presents a training performance score for the second user at the location, but does not compute a competition performance score for the second user because the second user has not engaged in any or a sufficient amount of competition activities at verified performance location 1.

The user device 120 is associated with a third user and presents a single mindfulness score for the third user and two performance scores for the third user. Each of the scores is associated with verified performance location 1 indicating that the verified performance location 1 is the only recognized location where the third user has engaged in performance activities. The mindfulness score computed for the third user is a performance-based mindfulness score that relates to the third user's state of mind during performance activities. Specifically, the third user has not been detected as engaging in any mindfulness activities and, thus, does not have a mindfulness score associated with mindfulness activities. However, the third user has been detected as engaging in competition-based performance activities at verified performance location 1 and the monitoring server 100 has been able to gather data to indicate the third user's mindfulness associated with the competition activities. In this case, the monitoring server 100 has computed a score of 85 to indicate that the third user has relatively good mindfulness during performance activities and likely does not suffer from performance anxiety. The third user also has training and competition scores computed for verified performance location 1. Both of these scores indicate a relatively high level of performance with the training score of 95 indicating a slightly higher level of training performance than the score of 90 for competition activities.

The user device 125 is associated with a fourth user and presents performance scores for the fourth user. As shown, the fourth user does not have any mindfulness scores, indicating that the fourth user has not engaged in any or a sufficient amount of mindfulness activities and has not engaged in competition activities that would lend itself to computation of a performance-based mindfulness score. As shown, the user has computed performance scores at inferred performance location 1 and inferred performance location 2. For inferred performance location 1, the fourth user has four scores computed, each being associated with a particular season with a spring score of 75, a summer score of 65, a fall score of 85, and a winter score of 70. As the scores indicate, the fourth user has been detected as having the highest level of performance at inferred performance location 1 during the fall with slightly lower performance in the spring and further reduced performance in the winter and summer. Because inferred performance location 1 was identified as a seasonal location with different scores for different seasons, the monitoring server 100 tracks user-specific performance scores for each of the various seasons related to inferred performance location 1. The fourth user also has a performance score of 60 associated with inferred performance location 2. Although inferred performance location 2 is also a seasonal location, the monitoring server 100 only generated a single score for inferred performance location 2 because that location is only operational in the summer.

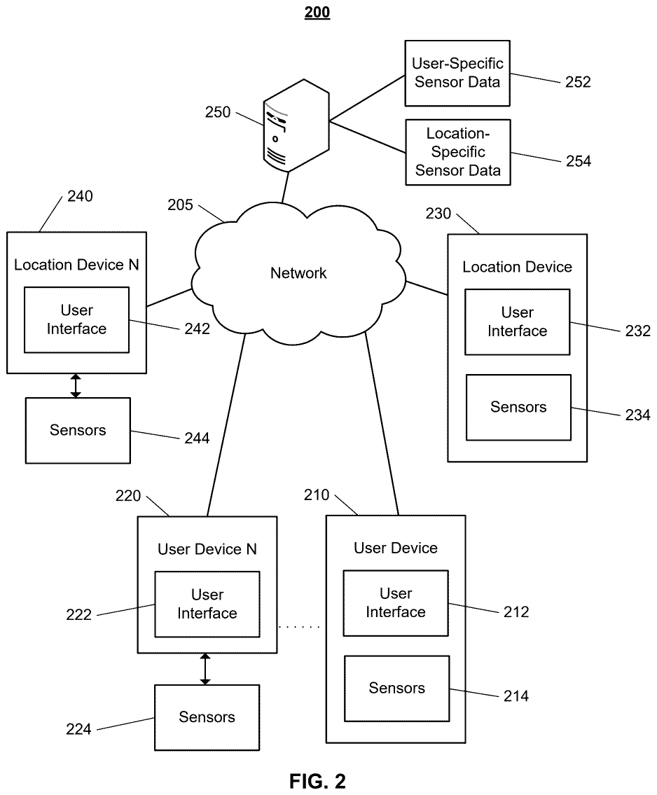

FIG. 2 illustrates an example of an electronic system 200 configured to perform location-based cardiovascular monitoring for performance and mindfulness sensing. As shown in FIG. 2, the system 200 includes a network 205, one or more user devices 210 and 220, one or more location devices 230 and 240, and a server 250 configured to communicate with the one or more user devices 210 and 220 and the one or more location devices 230 and 240 over the network 205.

The network 205 is configured to enable exchange of electronic communications between devices connected to the network 205. For example, the network 205 can be configured to enable exchange of electronic communications between the one or more devices 210 and 220, the one or more location devices 230 and 240, and the server 250. The network 205 can include, for example, one or more of the Internet, Wide Area Networks (“WANs”), Local Area Networks (“LANs”), analog or digital wired and wireless telephone networks (e.g., a public switched telephone network (“PSTN”), Integrated Services Digital Network (“ISDN”), a cellular network, and Digital Subscriber Line (“DSL”)), radio, television, cable, satellite, any other delivery or tunneling mechanism for carrying data, or a combination of these. The network 205 can include multiple networks or subnetworks, each of which can include, for example, a wired or wireless data pathway. The network 205 can include a circuit-switched network, a packet-switched data network, or any other network able to carry electronic communications (e.g., data or voice communications). For example, the network 205 can include networks based on the Internet protocol (“IP”), asynchronous transfer mode (“ATM”), the PSTN, packet-switched networks based on IP, X.25, or Frame Relay, or other comparable technologies and can support voice using, for example, voice over IP (“VoIP”), or other comparable protocols used for voice communications. The network 205 can include one or more networks that include wireless data channels and wireless voice channels. The network 205 can be a broadband network.

The one or more user devices 210 and 220 are devices that can present content, e.g., display user interfaces, and receive input. For instance, the device 210 may be a mobile device that hosts or runs one or more native applications. The mobile device 210 can be a cellular phone or a non-cellular locally networked device with a display. The mobile device 210 can include a cell phone, a smart phone, a tablet PC, a personal digital assistant (“PDA”), a smart watch, a ring sensor device, or any other portable device configured to communicate over a network and collect sensor data. The mobile device 210 can perform functions, such as placing personal telephone calls, exchanging text messages, playing music, playing video, displaying pictures, browsing the Internet, and maintaining an electronic calendar.

The mobile device 210 can include a user interface 212 and built-in sensors 214. The user interface 212 includes user interface components that allow a user to interact with the mobile device 210. For example, the user interface 212 may include a display (e.g., a touch-sensitive display) that displays content (e.g., text and graphics). The user interface 212 also may include a speaker to present audible output to the user. The user interface 212 further may include devices that allow a user to provide input to the mobile device 210, such as a touchscreen, physical or virtual buttons, and a microphone to receive voice input. The user interface 212 may be a mobile user interface with a graphical and touch-sensitive display that allows the user to interact with the device's applications, features, content, and functions.

The built-in sensors 214 may include any of the types of sensors described throughout this disclosure. For example, the built-in sensors 214 may include PPG sensors, ECG sensors, SPO2 monitors, pulse oximeters, etc. The built-in sensors 214 also may include neurological sensors that monitor and analyze neural activity in the brain or muscles and/or analyte sensors, such as glucose monitors, sweat sensors, electrochemical sensors, optical bio-sensors, piezoelectric bio-sensors, thermal bio-sensors, or any other types of sensors that are able to sense analytes in a person's body. The built-in sensors 214 further may include activity sensors that sense a user's activity level, such as accelerometers, gyroscopes, and magnetometers, and inertial measurement units (IMUs). In addition, the built-in sensors 214 may include location sensors, such as GPS sensors, beacon-based location sensors, triangulation-based location sensors, or any other types of sensors that can determine a user's location. The built-in sensors 214 may include environmental sensors that are configured to sense environmental conditions at a location of a user, such as noise sensors, light sensors, air temperature sensors, water temperature sensors, air quality sensors, wind speed sensors, water quality sensors, or any other types of environmental sensors that sense environmental conditions relevant to a user's performance or mindfulness.

The device 220 can be a mobile device similar to the device 210, or can be a general-purpose computer (e.g., a desktop personal computer, a workstation, or a laptop computer). The user interface 222 may be the same or similar to the user interface 212 and the sensors 224 may be the same or similar to the sensors 214. In examples in which the device 220 is a general-purpose computer, the user interface 222 may include user interface components used by general purpose computers (e.g., a larger display, an external display, a mouse, etc.) and/or the sensors 224 may include remote sensors that communicate with the device 220 over a wired or wireless connection. Although FIG. 2 illustrates two user devices for brevity, actual implementations can include more (and, perhaps, many more) or fewer devices.

The location device 230 may be similar to the user device 210 and the location device 240 may be similar to the user device 220. The location devices 230 and 240 may be any types of electronic devices that can receive or generate sensor related to the locations where the location devices 230 and 240 are situated and report the sensor data to the server 250. The sensors 234 and 244 may include environmental sensors that sense environmental attributes related to the locations where the location devices 230 and 240 are situated. For instance, the sensors 234 and 244 may include noise sensors, light sensors, air temperature sensors, water temperature sensors, air quality sensors, wind speed sensors, water quality sensors, or any other types of environmental sensors that sense environmental conditions relevant to a user's performance or mindfulness.

The server 250 is an electronic device configured to execute programs, access data from user-specific database 252, access data from location-specific database 254, and exchange communications with the multiple user devices 210 and 220 and the multiple location devices 230 and 240 over the network 205. In some examples, the server 250 gathers sensor data and stores it in the user-specific database 252 and the location-specific database 254. In these examples, the server 250 may process the stored sensor data to compute user-specific and location-specific performance and mindfulness scores. The server 250 also may analyze computed performance and mindfulness scores and make user-specific and location-specific recommendations to improve performance and mindfulness activities. The server 230 may include multiple servers or computers that communicate with each other and over network 205.

The user-specific database 252 is an electronic device configured to store user-specific data and exchange communications with the server 250 (e.g., multiple servers or computers) over a direct connection or network. For example, the user-specific database 252 may be configured to store data for user-specific performance and mindfulness and send stored data to the server 250 in response to requests (e.g., SQL statements or queries). The user-specific database 252 may store parts of a machine learning model used for generating user-specific performance and mindfulness scores from user-specific sensor data. The user-specific database 252 may include one or more databases and/or data warehouses.

The location-specific database 254 is an electronic device configured to store location-specific data and exchange communications with the server 250 (e.g., multiple servers or computers) over a direct connection or network. For example, the location-specific database 254 may be configured to store data for location-specific performance and mindfulness and send stored data to the server 250 in response to requests (e.g., SQL statements or queries). The location-specific database 254 may store parts of a machine learning model used for generating location-specific performance and mindfulness scores from location-specific sensor data. The location-specific database 254 may include one or more databases and/or data warehouses.

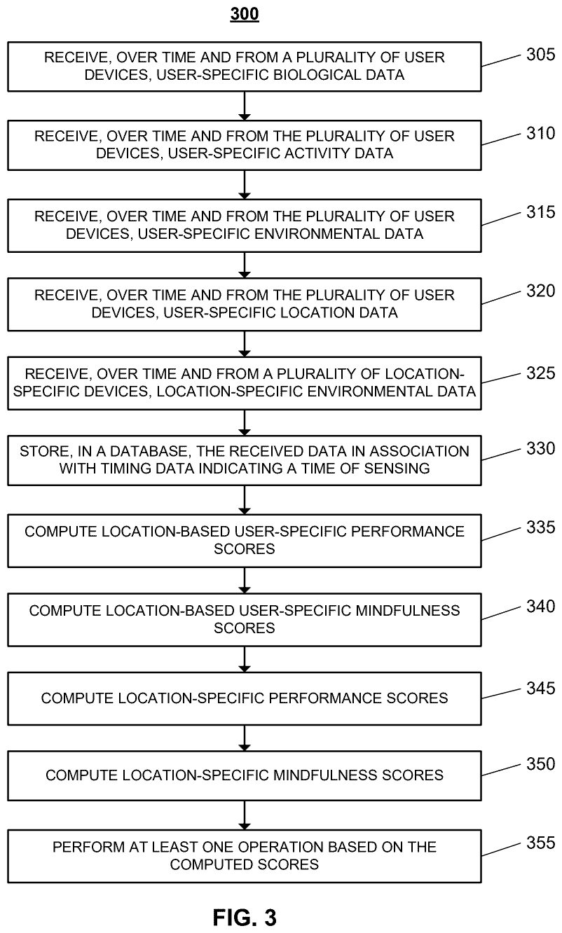

FIGS. 3-5, 7, 8, 10, 12, and 14 illustrate example processes. The operations of the example processes are described generally as being performed by the system 200. The operations of the example processes may be performed by one of the components of the system 200 (e.g., user device 210, location device 230, server 250, etc.) or may be performed by any combination of the components of the system 200. In some implementations, operations of the example processes may be performed by one or more processors included in one or more electronic devices.

FIG. 3 illustrates an example process 300 for performing location-based cardiovascular monitoring for performance and mindfulness sensing. The example process 300 begins when the system 200 receives, over time and from a plurality of user devices, user-specific biological data (305). For example, the system 200 receives user-specific biological data sensed by sensors associated with user devices that track biological data. The system 200 may continuously receive biological data throughout the course of the day as it is passively monitored without the user having to send the data to the system 200.

The system 200 also receives, over time and from the plurality of user devices, user-specific activity data (310). For instance, the system 200 receives user-specific activity data sensed by sensors associated with user devices that track activity data. The system 200 may continuously receive activity data throughout the course of the day as it is passively monitored without the user having to send the data to the system 200.

The system 200 further receives, over time and from the plurality of user devices, user-specific environmental data (315). For instance, the system 200 receives user-specific environmental data sensed by sensors associated with user devices that track environmental data. The system 200 may continuously receive environmental data throughout the course of the day as it is passively monitored without the user having to send the data to the system 200.

In addition, the system 200 receives, over time and from the plurality of user devices, user-specific location data (320). For instance, the system 200 receives user-specific location data sensed by sensors associated with user devices that track location data. The system 200 may continuously receive location data throughout the course of the day as it is passively monitored without the user having to send the data to the system 200.

The system 200 receives, over time and from a plurality of location-specific devices, location-specific environmental data (325). For instance, the system 200 receives location-specific environmental data sensed by sensors associated with location devices that track environmental data for corresponding locations. The system 200 may continuously receive environmental data throughout the course of the day as it is passively monitored by the sensors at the corresponding locations.

The system 200 stores, in a database, the received data in association with timing data indicating a time of sensing (330). For instance, the system 200 aggregates and stores the received data in the database in association with time stamps that indicate timing of when the specific data was sensed. The system 200 may aggregate all data together or may store each type of data separately. The system 200 also may group data by user and/or group data by location when it is stored in the database.

Based on the stored data in the database, the system 200 computes location-based user-specific performance scores (335) and location-based user-specific mindfulness scores (340). The system 200 may use any of the techniques described throughout this disclosure to compute the location-based user-specific performance and mindfulness scores.

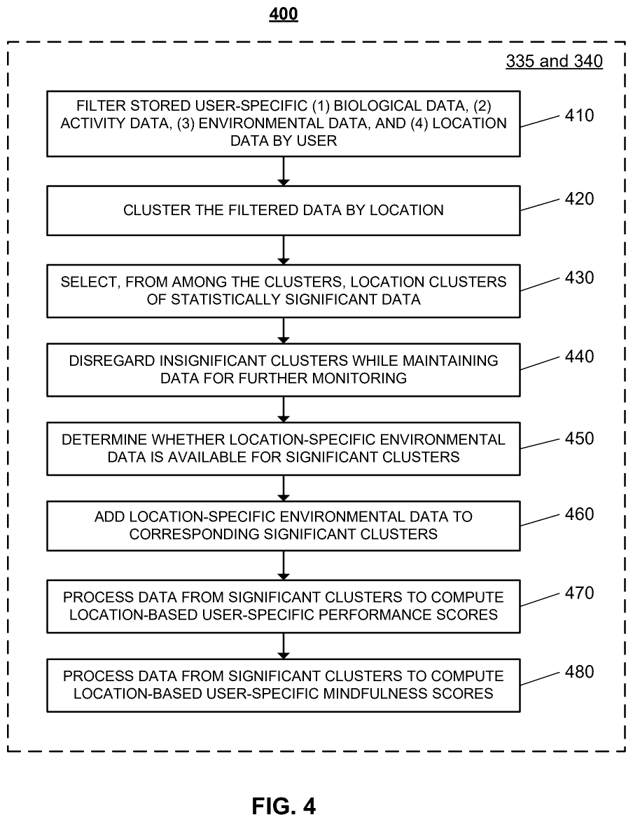

FIG. 4 illustrates an example process 400 for computing location-based user-specific performance and mindfulness scores. The process 400 may be performed as part of the operations described above with respect to reference numerals 335 and 340.

The process 400 begins by filtering stored user-specific (1) biological data, (2) activity data, (3) environmental data, and (4) location data by user (410). For example, the system 200 filters the stored data by specific users such that the system 200 is able to identify the stored data associated with each of the specific users. In this way, the system 200 filters the data to isolate the data for specific users such that the system is able to compute user-specific performance and mindfulness scores.

The system 200 clusters the filtered data by location (420). For instance, the system 200 clusters the filtered data into clusters of data that are each associated with a particular location. To cluster the filtered data, the system 200 may group sets of data together when the location data for the sets of data indicates a location that is within a threshold distance from one another. The system 200 may use machine learning to identify clusters of data associated with specific locations. In this way, the system 200 may learn how close two sets of data should be in terms of location to cluster them as being collected at the same location. For instance, the user may engage in performance and/or mindfulness events at a large venue at a particular location. In this case, the location for different events at the venue may be spaced apart at the venue, but within a sufficient distance to determine that it is actually associated with performance or mindfulness events that are happening at the larger venue. The system 200 may learn what different types of venues are used by the particular user and consider characteristics of those venues in its clustering process.

Based on clustering the filtered data by location, the system 200 selects, from among the clusters, location clusters of statistically significant data (430). The system 200 may analyze each location cluster to determine whether the cluster includes enough data to make a meaningful assessment of the user's performance or mindfulness at the corresponding location. The system 200 may require a certain number of days of performance or mindfulness data or a certain number of performance or mindfulness detected at the corresponding location.

The system 200 disregards insignificant clusters while maintaining data for further monitoring (440). For instance, the system 200 may drop the data associated with clusters of insignificant amounts of data for processing at this time. The system 200 does not delete the clusters of insignificant data, however, and retains the data for future clustering to the extent that additional data is sensed at the corresponding location to bring the amount of data at the location to a significant level.

The system 200 determines whether location-specific environmental data is available for significant clusters (450). The system 200 evaluates data received from location-specific devices that sense environmental data at specific locations and determines whether any environmental data has been received for each location corresponding to a significant cluster.

The system 200 adds location-specific environmental data to corresponding significant clusters (460). For instance, based on a determination that location-specific environmental data is available for significant clusters, the system 200 adds the location-specific environmental data to the corresponding cluster to increase the amount of data that can be analyzed to evaluate performance or mindfulness of the specific user at the corresponding location. The system 200 may compare the location-specific environmental data to the user-specific environmental data from the corresponding location to confirm whether the environmental data sensed at the location is consistent with the environmental data sensed by the user at the location.

The system 200 processes data from significant clusters to compute location-based user-specific performance scores (470) and to compute location-based user-specific mindfulness scores (480). The system 200 may use any of the techniques described throughout this disclosure to compute the location-based user-specific performance and mindfulness scores. For instance, the system 200 may detect performance and/or mindfulness events within the cluster of data, compute a performance and/or mindfulness score for each detected event, and compute overall performance and/or mindfulness scores for the user at the corresponding location by averaging the computed performance and/or mindfulness scores for the detected events. To compute a performance and/or mindfulness score for a detected event, the system 200 may consider biological data to assess the user's biographical state during the event, activity data to assess the user's activity state during the event, and environmental data to assess environmental conditions during the event. The system 200 may assign a biological score, an activity score, and an environmental score to each event and compute an event score based on the biological score, the activity score, and the environmental score (e.g., by averaging the scores or applying them to a formula with various weightings). The system 200 may assign each score using baselines related to typical biological data, activity data, and environmental data sensed for the corresponding type of event. For example, the system 200 may compare the specific user's data to various thresholds (e.g., heart rate thresholds, HRV thresholds, activity level thresholds, temperature thresholds, air quality thresholds, etc.) and assign the scores based on where the specific user's data fits within the various thresholds. The system 200 may apply different weightings to different types of events based on how important the data is for computing the relevant score. The system 200 also may apply the specific user's data to a formula that generates a score in the range of 0-100. In some examples, the system 200 may apply the specific user's data to a machine learning process that evaluates the specific user's data against a database of other users'data and generates a score based on how the specific user's data compares to the other users'data. In these examples, the system 200 may determine a percentile ranking for how the specific user's data compares to the other users'data and use the percentile ranking as the performance or mindfulness score.

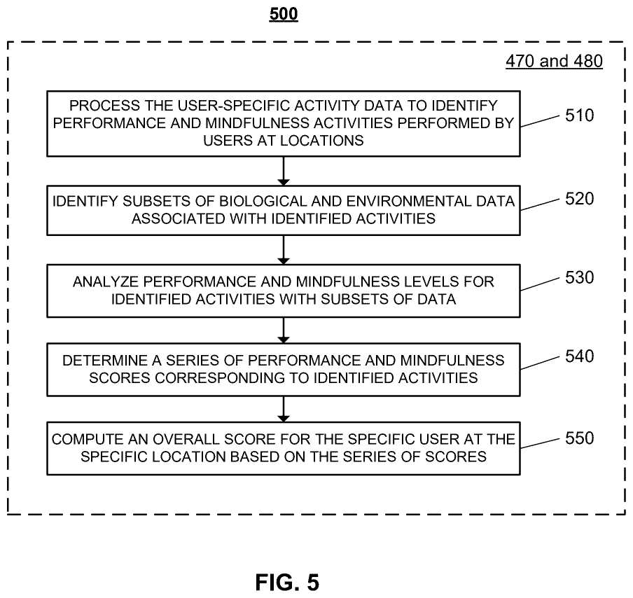

FIG. 5 illustrates an example process 500 for computing location-based user-specific performance and/or mindfulness scores. The process 500 may be performed as part of the operations described above with respect to reference numerals 470 and 480.

The process 500 begins by processing the user-specific activity data to identify performance and mindfulness activities performed by users at locations (510). For example, the system 200 may compare patterns of activity data tracked at the location corresponding to a cluster and determine whether the patterns of activity data match patterns of activity data for known activities. In this example, the system 200 may determine whether the patterns of activity data match patterns of activity for various types of events, such as walking events, running events, swimming events, mediation events, yoga events, etc. The system 200 may use machine learning to perform pattern matching to identify performance and mindfulness activities performed at the corresponding location.

The system 200 also identifies subsets of biological and environmental data associated with identified activities (520). For example, for each of the identified performance and mindfulness activities, the system 200 accesses the biological and environmental sensed at the corresponding location at a time of the identified activity. In this way, the system 200 gathers all of the data from the cluster that pertains to the activity identified and creates a subset of relevant data pertaining to the activity.

The system 200 analyzes performance and mindfulness levels for identified activities with subsets of data (530). For example, the system 200 analyzes the biological data, the activity data, and the environmental data to assess performance and/or mindfulness levels associated with the activity. In this example, the system 200 may analyze how the user's biological data for the activity compares to other users'biological data when engaging in the same type of activity and may analyze how the user's activity data for the activity compares to other users'activity data when engaging in the same type of activity. The system 200 also may consider the environmental data related to the activity and consider whether environmental conditions have an impact on the user's performance or mindfulness.

In some implementations, the system 200 determines a series of performance and mindfulness scores corresponding to identified activities (540). For instance, the system 200 may compute a performance and/or mindfulness score for each identified activity to generate a series of scores that reflect the user's performance and/or mindfulness for each activity detected at the corresponding location.

The system 200 computes an overall score for the specific user at the specific location based on the series of scores (550). In some examples, the system 200 averages the performance and/or mindfulness scores included in the series of scores that reflect the user's performance and/or mindfulness for each activity detected at the corresponding location. The system 200 also may apply the series of scores to a formula that weights the various scores and computes the overall performance and/or mindfulness using the formula. The system 200 further may provide the series of scores as input to a machine learning process that evaluates the series of scores and determines an overall score based on the series.

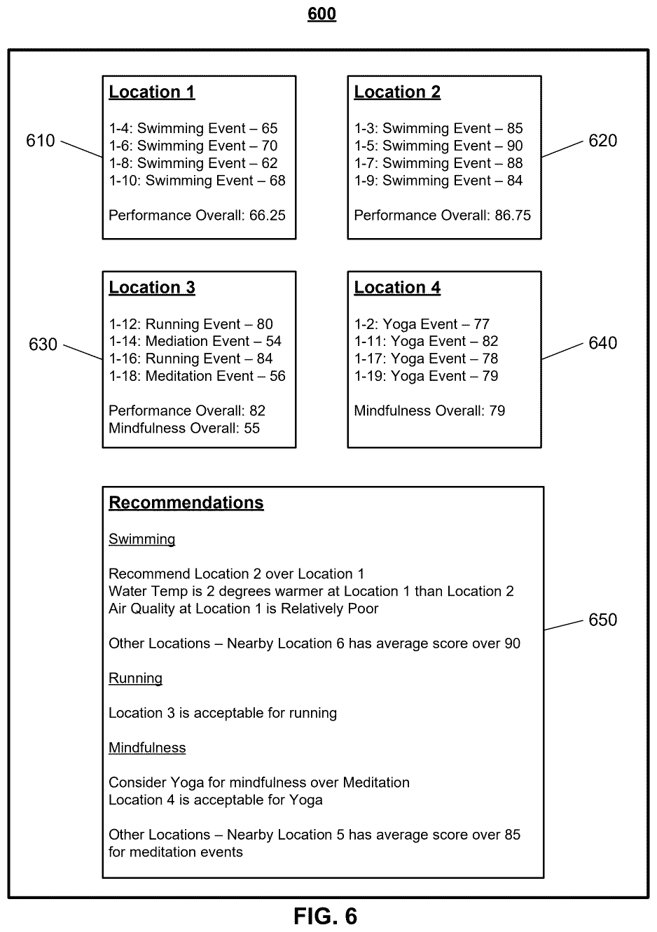

FIG. 6 illustrates an example user interface 600 related to computed location-based user-specific performance and/or mindfulness scores with associated recommendations generated by the system 200. The user interface 600 is a user-specific interface associated with performance and mindfulness activities of a particular user at different locations. The system 200 generates the user interface 600 using user-specific sensor data associated with the particular user, as well as location-specific sensor data associated with the locations where the particular user engages in performance and mindfulness activities.

The user interface 600 includes a first section 610 associated with a first location. As shown in the first section 610, the system 200 has received data for a series of swimming events that occurred on four different dates at location 1. As shown, the system 200 computed a score for each of the swimming events. The first swimming event was given a score of 65, the second swimming event was given a score of 70, the third swimming event was given a score of 62, and the fourth swimming event was given a score of 68. From the series of four scores, the system 200 computed an overall performance score of 66.25 by averaging the scores associated with each of the different swimming events.

The user interface 600 also includes a second section 620 associated with a second location. Similar to the first section 610, the second section 620 include scores for a series of swimming events and an overall performance score computed by averaging the scores associated with the series of events.

The user interface 600 further includes a third section 630 associated with a third location. As shown, the system 200 detected both performance events (e.g., running events) and mindfulness events (e.g., meditation events) at the third location. From the performance events, the system 200 computed an overall performance score for the third location by averaging the performance scores detected at the third location. From the mindfulness events, the system 200 computed an overall mindfulness score for the third location by averaging the mindfulness scores detected at the third location.

The user interface 600 also includes a fourth section 640 associated with a fourth location. As shown the system 200 detected mindfulness events, specifically yoga events, at the fourth location. The system 200 computed a mindfulness score associated with each of the yoga events, and then determined an overall mindfulness score associated with the fourth location by averaging the mindfulness scores computed for each of the different yoga events.

The user interface 600 includes a recommendation section 650 that displays recommendations automatically determined by the system 200 based on the performance and mindfulness scores shown in sections 610 to 640. In this example, the system 200 has organized the recommendations into a swimming section, a running section, and a mindfulness section. The system 200 organized the recommendations in this manner based on detection of swimming activities, running activities, and mindfulness activities occurring over the set of four locations. In the swimming recommendations, the system 200 recommends location 2 over location 1 for swimming because the overall performance score for location 2 is higher than the overall performance score for location 1. The system 200 also detected that the water temperature is 2 degrees warmer at location 1 than location 2, suggesting that the difference in water temperature may relate to the difference in performance scores. From this recommendation, the system 200 may automatically adjust water temperature settings at location 1 to match those of location 2 or may send a recommendation to a location-specific device associated with location 1 to suggest adjusting the water temperature by reducing it by 2 degrees. The system 200 also detected relatively poor air quality at location 1 as another indicator for why performance at location 1 is worse than performance at location 2. The system 200 also assessed other locations within a threshold distance of the locations where the particular user engages in performance and mindfulness activities, and compared the user-specific performance scores to scores associated with the other nearby locations that are within the threshold distance. As shown in section 650, the system 200 has detected a nearby location, location 6, that has an average score over 90 and offers a recommendation that the particular user try swimming at location 6 to see if the particular user has better performance at location 6 than, for example, location 1.

The running section of the recommendations simply notes that the system 200 has determined that location 3 is acceptable for running for the particular user. The system 200 made this recommendation by comparing the user-specific running scores at location 3 to a threshold established by scores of other users and determined that the particular user's performance in running events at location 3 is sufficiently strong (e.g., above the threshold) to continue running at location 3.

The mindfulness section has a recommendation that the particular user consider yoga for mindfulness over meditation. The system 200 arrived at this recommendation by noticing that the mindfulness scores for yoga events are better than the mindfulness scores for meditation events for the particular user and, thus, recommends that the particular user focus on yoga to support mindfulness. The system 200 also determined that location 4 is acceptable for yoga because the mindfulness scores for the particular user at location 4 are sufficiently strong relative to other user mindfulness scores at location 4 and other locations. The system 200 also assessed other nearby locations that are within a threshold distance of the locations where the user engages in mindfulness activities and identified location 5 as having an average score over 85 for meditation events. Based on recognition that location 5 has a relatively high score for meditation events (as compared to the particular user's scores), the system 200 determined to recommend that the particular user consider location 5 for meditation since the particular user has relatively lower scores for meditation events at location 3. In this way, the system 200 is able to guide the particular user to a different location where the particular user may have more success with meditation events, thereby improving the particular user's mindfulness while providing concrete data to suggest that the user may want to consider a change in location for meditation events.

Returning to FIG. 3, in some implementations, the system 200 computes location-specific performance scores (345) and computes location-specific mindfulness scores (350). The system 200 may use any of the techniques described throughout this disclosure to compute the location-specific performance and mindfulness scores.

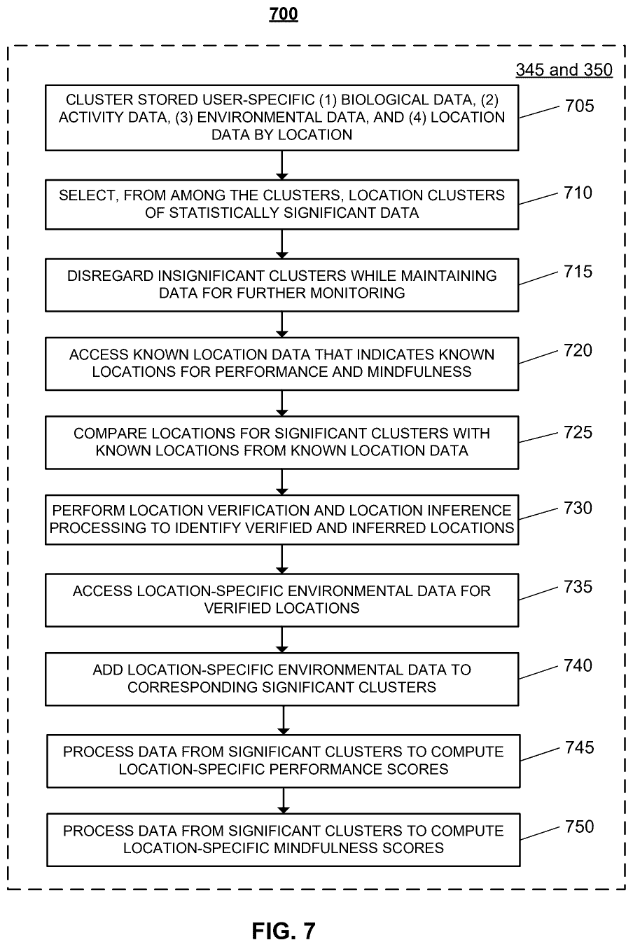

FIG. 7 illustrates an example process 700 for computing location-specific performance and mindfulness scores. The process 700 may be performed as part of the operations described above with respect to reference numerals 345 and 350.

The system 200 clusters stored user-specific (1) biological data, (2) activity data, (3) environmental data, and (4) location data by location (705). For instance, the system 200 uses techniques similar to those discussed above with respect to operation 420 to cluster the stored user-specific (1) biological data, (2) activity data, (3) environmental data, and (4) location data. In this case, the stored data is not filtered into specific users and the system 200 performs clustering of all data collected at each location. In this regard, the clustering by location allows the system 200 to develop data to assess specific locations generally. In some examples, the system 200 limits the time period of the data before clustering to ensure the data reflects current circumstances for each location and is not overly skewed based on older data.

The system 200 then selects, from among the clusters, location clusters of statistically significant data (710). For example, the system 200 uses techniques similar to those discussed above with respect to operation 430 to select clusters of statistically significant data. In this case, the system 200 may ensure that the clusters include data from a sufficient number of users and/or a sufficient number of different types of users to ensure the system 200 is able to discern the location's impact on performance and/or mindfulness.

The system 200 disregards insignificant clusters while maintaining data for further monitoring (715). For instance, the system 200 uses techniques similar to those discussed above with respect to operation 440 to disregard insignificant clusters while maintaining the disregarded data for further monitoring.

The system 200 accesses known location data that indicates known locations for performance and mindfulness (720). For example, the system 200 accesses, from electronic storage, a list of known locations for performance and mindfulness. In this example, the list may include the name of each known location, the types of activities associated with each known location, and location information (e.g., an address) for each known location. The known location data may include data used to generate a map with performance and mindfulness locations indicated or may include data used to generate a directory of performance and mindfulness locations.

The system 200 compares locations for significant clusters with known locations from known location data (725). For instance, the system 200 compares location information (e.g., an address or GPS coordinates) of clustered locations with the location information (e.g., addresses or GPS coordinates) of locations listed in the known location data to determine whether any of the clustered locations match known locations.

The system 200 performs location verification and location inference processing to identify verified and inferred locations (730). For instance, the system 200 uses the comparison results from operation 725 to determine whether each significant cluster corresponds to a verified location in the known location data or not. The system 200 may determine whether the location information in the known location data matches the location of a significant cluster and also may determine the types of activities detected within the significant cluster match the types of activities set forth in the known location data for a matching location. If the location and the types of activities match exactly or within a threshold level of similarity, the system 200 determines that the significant cluster corresponds to a verified location. If not, the system 200 determines that the significant cluster corresponds to an inferred location. The system 200 may develop confidence scores to reflect how confident the system 200 is in identifying the significant cluster as a verified location or an inferred location and may take different approaches for the significant cluster based on the confidence score. For instance, if the confidence score for the significant cluster being a verified location is above a threshold, the system 200 may automatically add the significant cluster as a verified location. If the confidence score for the significant cluster being a verified location is lower than the threshold, the system 200 may query the user to confirm whether the significant cluster is a verified location or not.

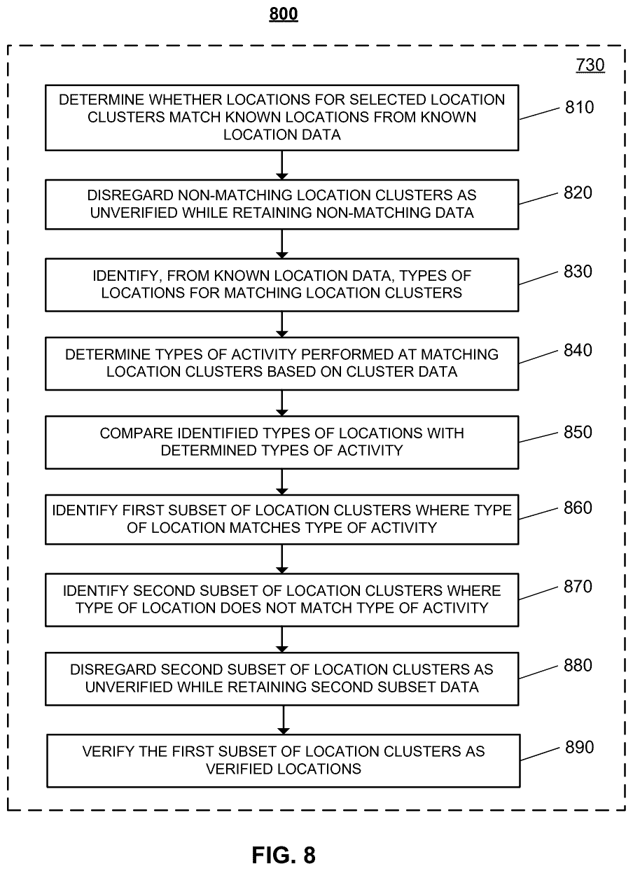

FIG. 8 illustrates an example process 800 for performing location verification processing to identify verified locations. The process 800 may be performed as part of the operations described above with respect to reference numeral 730.

The system 200 determines whether locations for selected location clusters match known locations from known location data (810). For instance, the system 200 compares location information (e.g., addresses or GPS coordinates) for the selected location clusters with location information (e.g., addresses or GPS coordinates) in the known location data to determine whether a match exists. The system 200 may determine that a match exists when the location information matches exactly or within a threshold distance from one another.

The system 200 disregards non-matching location clusters as unverified while retaining non-matching data (820). For instance, the system 200 may drop the data associated with non-matching location clusters for processing at this time. The system 200 does not delete the non-matching location clusters, however, and retains the data for future processing to the extent that the corresponding location becomes verified at a later point in time.

The system 200 identifies, from known location data, types of locations for matching location clusters (830). For instance, for the matching locations, the system 200 identifies the type of the location from the known location data. The system 200 may determine whether the matching location is a pool, a track, a gym, a meditation center, a yoga studio, etc. The system 200 also may access a list of activities that are performed at the matching location.

The system 200 determines types of activity performed at matching location clusters based on cluster data (840). For instance, the system 200 analyzes the biological data and the activity data within the matching location clusters to determine what types of activities users have been engaging in at the location corresponding to the matching cluster. The system 200 may compare patterns of biological and/or activity data tracked at the location corresponding to the matching cluster and determine whether the patterns of biological and/or activity data match patterns of biological and/or activity data for known activities. In this example, the system 200 may determine whether the patterns of activity data match patterns of activity for various types of events, such as walking events, running events, swimming events, mediation events, yoga events, etc. The system 200 may use machine learning to perform pattern matching to identify performance and mindfulness activities performed at the location corresponding to the matching cluster.

The system 200 compares identified types of locations with determined types of activity (850). For instance, the system 200 compares the types of activities that occur at the identified types of locations with the determined types of activities performed at matching location clusters.

The system 200 identifies a first subset of location clusters where type of location matches type of activity (860). For instance, based on the comparison of the types of activities that occur at the identified types of locations with the determined types of activities performed at matching location clusters, the system 200 identifies a first subset of location clusters where the types of activities match.

The system 200 identifies a second subset of location clusters where type of location does not match type of activity (870). For instance, based on the comparison of the types of activities that occur at the identified types of locations with the determined types of activities performed at matching location clusters, the system 200 identifies a second subset of location clusters where the types of activities do not match.

The system 200 disregards second subset of location clusters as unverified while retaining second subset data (880). For instance, because the types of activities do not match for the second subset of location clusters, the system 200 determines that the second subset of location clusters are unverified given the discrepancy with the known location data. The system 200 still retains the second subset of data for future processing since the types of activities may change to matching activities based on additional sensed data. Additionally, the known location data may require updates based on changes in known locations (e.g., a gym changing to a yoga studio) and, with changes to the known location data, the types of activities may end up matching the updated known location data.

The system 200 verifies the first subset of location clusters as verified locations (890). For instance, the system 200 designates the first subset of location clusters as verified locations because the location information and the types of activities match the known location data with a relatively high degree of confidence (e.g., a confidence score above a threshold). Upon verification, the system 200 evaluates the first subset of location clusters to compute scores and make recommendations for the verified locations. The system 200 also may establish contact with systems at the verified locations to alert an owner or manager of the verified locations that the system 200 has captured a significant amount of performance and/or mindfulness data that is relevant to the verified locations.

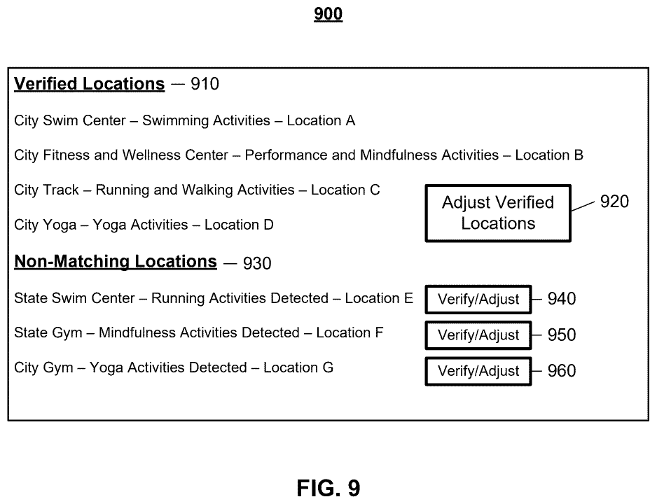

FIG. 9 illustrates an example user interface 900 used for location verification. The user interface 900 includes a first section 910 that displays verified locations. The first section 910 displays a listing of four verified locations with each listing including the name of the location, the types of activities detected at the location, and location information (e.g., an address) associated with the location. The user interface 900 also includes a button 920 that allows the user to adjust the verified locations. Upon receipt of user input activating the button 920, the system 200 may display another user interface or additional controls on the user interface 900 that enable the user to adjust the verified locations. The adjustments may include removing the verified location designation for any of the locations listed and changing the name, types of activities, and location information for any of the locations listed.

The user interface 900 also includes a second section 930 that displays non-matching locations that have not been verified. The non-matching locations have not been verified because the location was not included in known location data and/or the name, types of activities, and/or location information does not match data in the known location data. The second section 930 displays a listing of three non-matching locations with each listing including the name of the location, the types of activities detected at the location, and location information (e.g., an address) associated with the location. The second section 930 also includes buttons 940, 950, and 960 with each button being associated with a corresponding non-matching location. Activation of one of the buttons 940, 950, and 960 may verify the corresponding location and move the location from the listing of non-matching locations to the listing of verified locations. The system 200 also may update the known location data based on user input verifying a non-matching location. The buttons 940, 950, and 960 also may be used to make adjustments to the corresponding non-matching location, including changing the name, types of activities, and location information for the corresponding location.

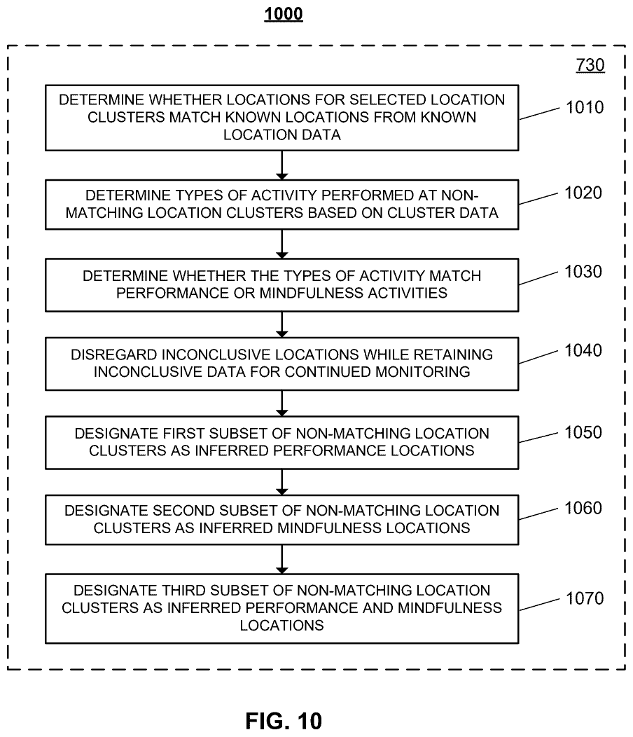

FIG. 10 illustrates an example process 1000 for performing location inference processing to identify inferred locations. The process 1000 may be performed as part of the operations described above with respect to reference numeral 730.

The system 200 determines whether locations for selected location clusters match known locations from known location data (1010). For example, the system 200 uses techniques similar to those discussed above with respect to operation 810 to determine whether locations for selected location clusters match known locations from known location data.

The system 200 determines types of activity performed at non-matching location clusters based on cluster data (1020). For example, the system 200 uses techniques similar to those discussed above with respect to operation 840 to determine types of activity performed at non-matching location clusters based on cluster data.

The system 200 determines whether the types of activity match performance or mindfulness activities (1030). For example, the system 200 analyzes the determined types of activity performed at non-matching location clusters and classifies them as performance or mindfulness activities. In this example, the system 200 may classify swimming, running, walking, and weightlifting activities as performance activities and may classify meditation and yoga activities as mindfulness activities.

The system 200 disregards inconclusive locations while retaining inconclusive data for continued monitoring (1040). If the activity types cannot be determined or the types of activities are not able to be classified as performance or mindfulness activities, the system 200 designates the corresponding locations as having inconclusive data. In this case, the system 200 may drop the data associated with inconclusive locations for processing at this time. The system 200 does not delete the data associated with inconclusive locations, however, and retains the data for future processing to the extent that additional data is sensed at the inconclusive locations to enable conclusive results for one or more of the inconclusive locations.

The system 200 designates a first subset of non-matching location clusters as inferred performance locations (1050). Based on the determination of activities as being performance activities for a first subset of the non-matching location clusters, the system 200 designates the first subset of non-matching location clusters as inferred performance locations. Based on this designation, the system 200 processes the first subset of the non-matching location clusters as inferred performance locations moving forward.

The system 200 designates a second subset of non-matching location clusters as inferred mindfulness locations (1060). Based on the determination of activities as being mindfulness activities for a second subset of the non-matching location clusters, the system 200 designates the second subset of non-matching location clusters as inferred mindfulness locations. Based on this designation, the system 200 processes the second subset of the non-matching location clusters as inferred mindfulness locations moving forward.

The system 200 designates a third subset of non-matching location clusters as inferred performance and mindfulness locations (1070). Based on the determination of activities as being performance and mindfulness activities for a third subset of the non-matching location clusters, the system 200 designates the third subset of non-matching location clusters as inferred performance and mindfulness locations. Based on this designation, the system 200 processes the third subset of the non-matching location clusters as inferred performance and mindfulness locations moving forward.

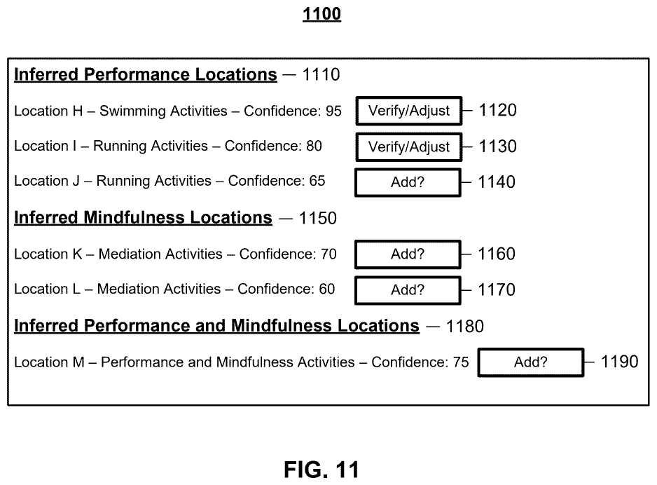

FIG. 11 illustrates an example user interface 1100 used for location inference processing. The user interface 1100 includes a first section 1110 that lists inferred performance locations. As shown, the first section 1110 lists three inferred performance locations. Each inferred location includes location information (e.g., an address), the types of activities detected at the location, and a confidence score that indicates how confident the system 200 is in inferring the location. The system 200 determines the confidence score by analyzing how closely the characteristics of the inferred location match locations known to be performance locations. The system 200 may consider the percentage of activity profiles that match performance events at the inferred location, the percentage of users that engage in at least performance event when visiting the inferred location, how closely the environmental data sensed at the inferred location matches environmental data associated with known performance locations, how confidently each type of sensor data (e.g., biological and activity sensor data) matches profiles associated with a performance location, and/or any other statistics that give the system 200 confidence that the inferred location is a location where performance events occur. The system 200 may use machine learning techniques to determine the confidence score by adaptively learning over time how confident the system 200 can be in inferring performance locations.

The first section 1110 includes buttons 1120, 1130, and 1140 that each correspond to one of the inferred locations. Because the first two locations in the first section 1110 have relatively high confidence scores (e.g., more than a threshold of 80 or above), the first two locations have been automatically added by the system 200 as inferred performance locations. Accordingly, the buttons 1120 and 1130 can be used to verify the inferred locations and/or adjust the location information, types of activities, and/or add a name for the inferred performance location. For example, a user can activate the button 1120 to add the first location in the first section 1110 to a list of verified performance locations. In this example, upon activation of the button 1120, the system 200 also adds the first location in the first section 1110 to the known location data associated with verified performance locations.

The button 1140 associated with the third location in the first section 1110 is associated with a location with a lower confidence score of 65 and, as a consequence, was not added automatically as an inferred performance location. To add the third location to the list of inferred performance locations tracked by the system 200, a user activates the button 1140 to add the third location as in an inferred performance location.

The user interface 1100 also includes a second section 1150 that lists inferred mindfulness locations. The second section 1150 is similar to the first section 1110, but lists mindfulness locations instead of performance locations. The buttons 1160 and 1170 are similar to the button 1140 because they associated with inferred mindfulness locations where the confidence score is lower than the threshold (e.g., lower than a confidence score of 80) and allow the user to add the corresponding locations as inferred mindfulness locations that are tracked by the system 200.

The user interface 1100 further includes a third section 1180 that lists inferred performance and mindfulness locations. The third section 1180 is similar to the first section 1110 and the second section 1150, but lists locations that have been identified as being both performance and mindfulness locations. The button 1190 is similar to the button 140 because it is associated with an inferred performance and mindfulness location where the confidence score is lower than a threshold (e.g., lower than a confidence score of 80) and allows the user to add the corresponding location as an inferred performance and mindfulness location that is tracked by the system 200.

Returning to FIG. 7, the system 200 accesses location-specific environmental data for verified locations (735) and adds the location-specific environmental data to corresponding significant clusters (740). Based on verification of the verified locations, the system 200 checks whether the verified locations have provided location-specific environmental data sensed by sensors at the verified locations. When location-specific environmental data has been received, the system 200 adds it to the clustered data associated with the verified locations. The added location-specific environmental data aids the system 200 in assessing how environmental conditions at the location impacts performance and/or mindfulness.

The system 200 processes data from significant clusters to compute location-specific performance scores (745) and processes data from significant clusters to compute location-specific mindfulness scores (750). The system 200 may use any of the techniques described throughout this disclosure to compute the location-specific performance and mindfulness scores. For instance, the system 200 may detect performance and/or mindfulness events for all users within the cluster of data, compute a performance and/or mindfulness score for each detected event, and compute overall performance and/or mindfulness scores for the corresponding location by averaging the computed performance and/or mindfulness scores for the detected events. To compute a performance and/or mindfulness score for a detected event, the system 200 may consider biological data to assess the user's biographical state during the event, activity data to assess the user's activity state during the event, and environmental data to assess environmental conditions during the event. The system 200 may assign a biological score, an activity score, and an environmental score to each event and compute an event score based on the biological score, the activity score, and the environmental score (e.g., by averaging the scores or applying them to a formula with various weightings). The system 200 may assign each score using baselines related to typical biological data, activity data, and environmental data sensed for the corresponding type of event. For example, the system 200 may compare the user data to various thresholds (e.g., heart rate thresholds, HRV thresholds, activity level thresholds, temperature thresholds, air quality thresholds, etc.) and assign the scores based on where the user data fits within the various thresholds. The system 200 may apply different weightings to different types of data based on how important the data is for computing the relevant score. The system 200 also may apply the user data to a formula that generates a score in the range of 0-100. In some examples, the system 200 may apply the user data for all users at the location to a machine learning process that evaluates the user data against a database of different location data and generates a score based on how the user data at the location compares to data collected at the different locations. In these examples, the system 200 may determine a percentile ranking for how the corpus of data at the location compares to the corpus of data at the different locations and use the percentile ranking as the performance or mindfulness score.

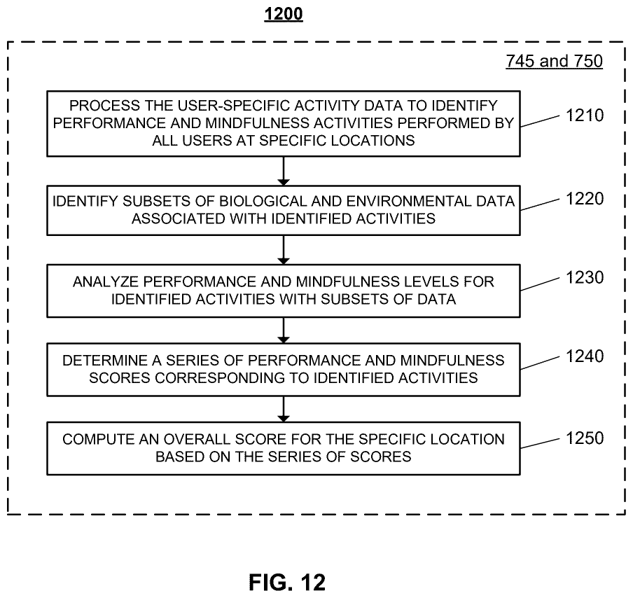

FIG. 12 illustrates an example process 1200 for computing location-specific performance and mindfulness scores. The process 1200 may be performed as part of the operations described above with respect to reference numerals 745 and 750.

The system 200 processes the user-specific activity data to identify performance and mindfulness activities performed by all users at specific locations (1210). For example, the system 200 uses techniques similar to those discussed above with respect to operation 510 to process the user-specific activity data to identify performance and mindfulness activities performed by all users at specific locations. Unlike the operation 510, the system 200 here processes data for all users collected at the location.

The system 200 identifies subsets of biological and environmental data associated with identified activities (1220). For example, the system 200 uses techniques similar to those discussed above with respect to operation 520 to identify subsets of biological and environmental data associated with identified activities. Unlike the operation 520, the system 200 here processes data for all users collected at the location.

The system 200 analyzes performance and mindfulness levels for identified activities with subsets of data (1230). For example, the system 200 uses techniques similar to those discussed above with respect to operation 530 to analyze performance and mindfulness levels for identified activities with subsets of data. Unlike the operation 530, the system 200 here processes data for all users collected at the location.

The system 200 determines a series of performance and mindfulness scores corresponding to identified activities (1240). For example, the system 200 uses techniques similar to those discussed above with respect to operation 540 to determine a series of performance and mindfulness scores corresponding to identified activities. Unlike the operation 540, the system 200 here processes data for all users collected at the location.

The system 200 computes an overall score for the specific location based on the series of scores (1250). For example, the system 200 uses techniques similar to those discussed above with respect to operation 550 to compute an overall score for the specific location based on the series of scores. Unlike the operation 550, the system 200 here processes data for all users collected at the location and computes a score for the location in general, rather than a score for a specific user at the location.

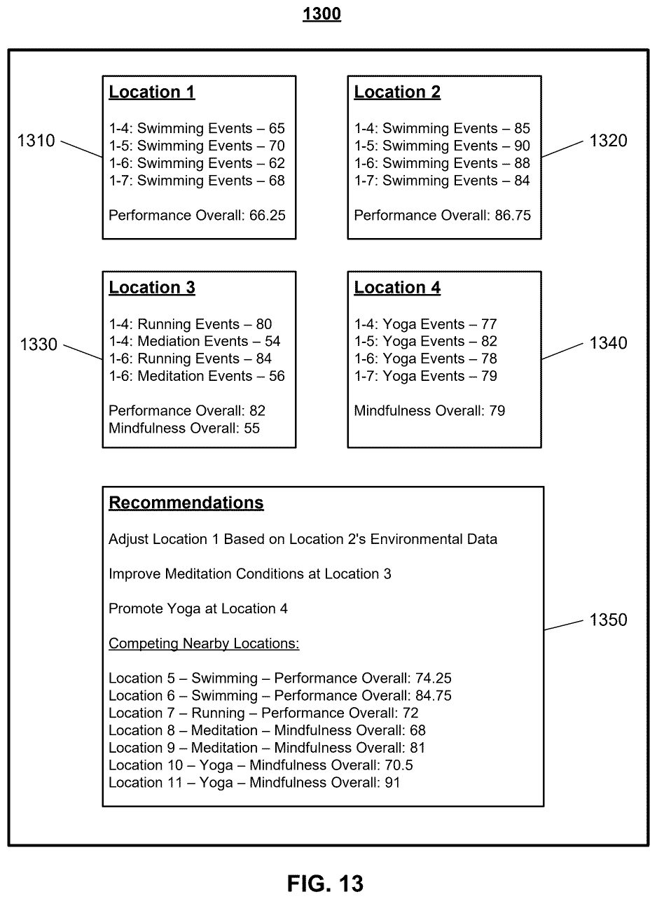

FIG. 13 illustrates an example user interface 1300 related to computed location-based performance and/or mindfulness scores with associated recommendations generated by the system 200. The user interface 1300 is a location-specific interface associated with performance and mindfulness activities of different locations (e.g., different locations owned or managed by a particular owner or group). The system 200 generates the user interface 1300 using location-specific sensor data associated with the particular locations, accounting for data associated with all users engaging in performance and/or mindfulness activities at the locations.

The user interface 1300 includes a first section 1310 associated with a first location. As shown in the first section 1310, the system 200 detected swimming events occurring at the first location on four consecutive days in January. On each of these days, the system 200 tracked multiple swimming events from multiple users taking place at the first location and generated a score associated with the swimming events that occurred on that day. As shown, the system 200 computed a score of 65 for the swimming events that occurred on January 4, a score of 70 for the swimming events that occurred on January 5, a score of 62 for the swimming events that occurred on January 6, and a score of 68 for the swimming events that occurred on January 7. The system 200 then computed an overall performance score of 66.25 as an average of the daily scores for swimming events that took place at the first location.

The user interface 1300 also includes a second section 1320. The second section 1320 is similar to the first section 1310, but includes performance scores computed at a second location that is different than the first location.

The user interface 1300 further includes a third section 1330 associated with a third location. The third section 1330 includes performance scores for running events that occurred on two days in the month of January, and also includes mindfulness scores for meditation events that occurred on the same two days in the month of January. The system 200 used the scores for the running events to compute an overall performance score for the third location and used the scores for the meditation events to commute an overall mindfulness score for the third location.

The user interface 1300 also includes a fourth section 1340 associated with a fourth location. The fourth section 1440 includes mindfulness scores for yoga events that occurred on four consecutive days in the month of January. The system 200 used the mindfulness scores for the yoga events to compute an overall mindfulness score for the fourth location. For instance, the system 200 averaged the scores computed on each day in the month of January for yoga events to compute the overall mindfulness score for the fourth location.

In addition, the user interface 1300 includes a recommendation section 1350 that displays recommendations automatically determined by the system 200 based on the performance and mindfulness scores shown in the first, second, third, and fourth sections 1310 to 1340. The recommendations are presented to an owner or manager that is associated with all of the four locations shown in the interface 1300. As shown, the first recommendation is to adjust environmental settings at the first location based on environmental data associated with the second location. As shown by a comparison of the performance scores in the first section 1310 to the performance scores in the second section 1320, the second location had higher performance scores associated with swimming events. Based on detecting that the second location had higher performance scores, the system 200 automatically identified adjustments to the first location that could be helpful in improving the performance scores for swimming events at the first location. Specifically, the system 200 determined that the environmental conditions at the second location are different than the environmental conditions at the first location and determined that the difference in environmental conditions might be related to the difference in performance scores. Based on these determinations, the system 200 automatically determined to recommend that the owner or manager adjust the environmental conditions at the first location to match those at the second location to see whether that would improve the performance scores at the first location.

The second recommendation in the recommendation section 1350 recommends that the owner or manager improve the meditation conditions at the third location. The system 200 automatically determined to make this recommendation because the mindfulness scores for meditation events occurring at the third location are relatively low (e.g., below a threshold of 60). By detecting relatively low mindfulness scores for meditation events occurring at the third location, the system 200 automatically determined that the meditation conditions at the third location should be improved. The threshold used for determining when to recommend improving conditions at a particular location may be based on data at similar types of locations and the mindfulness scores for meditation events at those similar types of locations. In this way, the system 200 assesses the relative mindfulness scores for meditation events at various locations and suggests improvements where the mindfulness scores for a particular location, such as the third location, fall behind peer locations.

The third recommendation in the recommendation section 1350 suggests promoting yoga at the fourth location. In this case, the system 200 determined that the mindfulness scores associated with yoga events are relatively high at the fourth location and, as a consequence, the fourth location should be promoted as one where users should engage in yoga activities. The system 200 made the determination to promote yoga at the fourth location based on a determination that the mindfulness scores for yoga events at the fourth location are above a threshold (e.g., above a threshold of 75). Again, similar to the recommendation to improve meditation conditions at the third location, the system 200 may take into account relative mindfulness scores for yoga events at similar types of locations and recommend promoting yoga at the fourth location when the mindfulness scores for yoga events at the fourth location are better than peer locations.

The recommendation section 1350 also lists performance and mindfulness scores for competing nearby locations. For example, the system 200 automatically determines locations that are within a threshold distance of the four locations listed in the user interface 1300. In this example, the system 200 determines performance or mindfulness scores associated with the nearby locations and presents them to the owner or manager for consideration. In this way, the owner or manager is able to see how well his or her locations are doing relative to competing nearby locations to enable the owner or manager to determine whether improvements to his or her locations are needed.

Returning to FIG. 3, the system 200 performs at least one operation based on the performance and mindfulness computed scores (355). The system 200 may perform any of the techniques described throughout this disclosure based on the computed performance and mindfulness scores. For example, the system 200 may display computed performance and mindfulness scores to users and assess the performance and mindfulness scores to make user recommendations for locations where specific users achieve superior performance and/or mindfulness. The system 200 also may make location recommendations that assist locations in improving performance and/or mindfulness scores or may automatically adjust environmental settings at locations to improve performance and/or mindfulness scores.

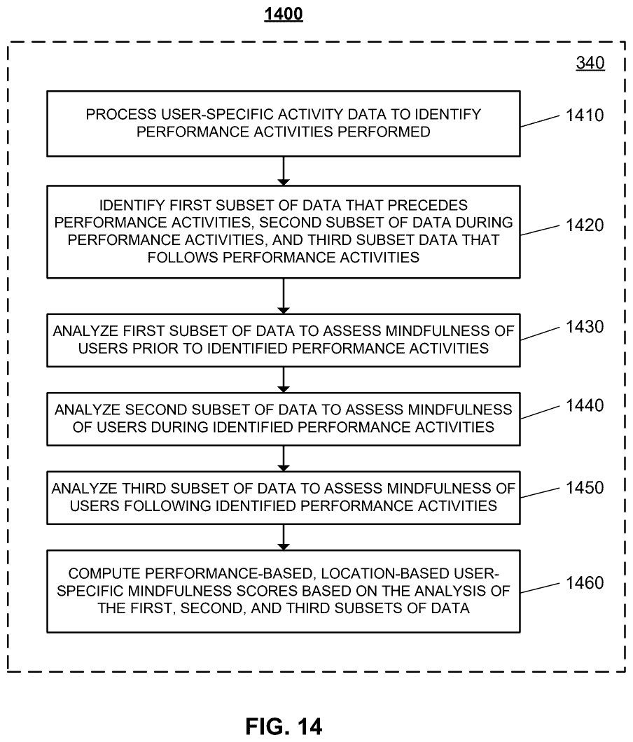

FIG. 14 illustrates an example process 1400 for computing performance-based, location-based user-specific mindfulness scores. The process 1400 may be performed as part of the operations described above with respect to reference numeral 340.

The system 200 processes user-specific activity data to identify performance activities performed (1410). For example, the system 200 analyzes the activity data to identify competition activities performed by a specific user. The system 200 may identify competition activities by identifying instances of higher intensity performance activities. In some examples, the system 200 compares data from several users at the location to determine when competitions are taking place. In these examples, the system 200 determines that a relatively large number of users are engaging in relatively high intensity performance activities at around the same time, suggesting that competition is taking place. For instance, the system 200 may identify that multiple swimmers are at a pool and are engaging in high intensity swimming activities for short periods of time with gaps of rest in between the periods of high intensity swimming activity. In this case, the system 200 is able to discern that the swimmers are participating in a swim meet and competing in events taking place throughout the day. With this recognition, the system 200 is able to isolate events that are competed in by specific users as competition events.

The system 200 identifies a first subset of data that precedes performance activities, a second subset of data during performance activities, and a third subset data that follows performance activities (1420). Upon isolation of the competition events identified for the specific user, the system 200 identifies a first subset of data that proceeds the competition events, a second subset of data that was collected during the competition events, and a third subset of data that follows the competition events. Because the system 200 was able to identify the competition event (e.g., a swimming race at a swim meet), the system 200 is able to identify subsets of the data before, during, and after the competition event. The system 200 may consider data within a threshold period of time before and after the competition events (e.g., data within 5 or 10 minutes of the competition event).

The system 200 analyzes the first subset of data to assess mindfulness of users prior to identified performance activities (1430). The system 200 analyzes the biological data for the user and the activity data for the user prior to the competition events. For instance, the system 200 may assess whether the user has a relatively high heart rate prior to the competition events, and/or whether the activity data indicates that the user was jittery prior to the competition events. In this way, the system 200 analyzes the first subset of data to determine whether the user is nervous or experiencing anxiety prior to the competition events.

The system 200 analyzes the second subset of data to assess mindfulness of users during identified performance activities (1440). The system 200 also analyzes the second subset of data to assess mindfulness during the competition events. In this way, the system 200 determines whether nervousness or anxiety continues throughout the event or whether the user is able to perform at a higher level of mindfulness during the event.

The system 200 analyzes the third subset of data to assess mindfulness of users following identified performance activities (1450). The system 200 further analyzes the third subset of data to assess the user's mindfulness following the competition events. The system 200 uses techniques similar to those used to process the first subset of data described with respect to operation 1430, and assesses whether the user experiences mindfulness issues following a competition event.

The system 200 computes performance-based, location-based user-specific mindfulness scores based on the analysis of the first, second, and third subsets of data (1460). Based on analysis of the first, second, and third subsets of data, the system 200 computes performance-based location-based user-specific mindfulness scores that indicate the specific user's mindfulness during competition events at different locations. The system 200 may compute separate scores for pre-competition, during competition, and post-competition. The system 200 also may compute an overall performance-based mindfulness score for the specific user at the specific location that accounts for the analysis of the first, second, and third subsets of data. For instance, the system 200 may average pre-competition, during competition, and post-competition performance-based mindfulness scores to generate the overall performance-based mindfulness score or may apply the pre-competition data, during competition data, and post-competition data to a formula to generate a score between 0 and 100 that reflects the specific user's overall mindfulness at the specific location.

In some implementations, the system 200 then analyzes the performance-based mindfulness scores to make recommendations to the specific user. For instance, the system may determine that the user's performance-based mindfulness is better at specific locations and make recommendations that the user attempt to take part in competition events at the specific locations where the user's performance-based mindfulness is better. The system 200 also may analyze environmental conditions at different locations, and how the environmental conditions relate to the user's performance-based mindfulness. From this analysis, the system 200 can determine what environmental conditions lead to good performance-based mindfulness for the specific user and suggest that the user attempt to compete at locations with those attributes and/or to adjust attributes at locations to match attributes where the user has better performance-based mindfulness. The system 200 further may process the performance-based mindfulness scores to attempt to determine whether the specific user is suffering from performance anxiety, and would benefit from treatment related to performance anxiety.

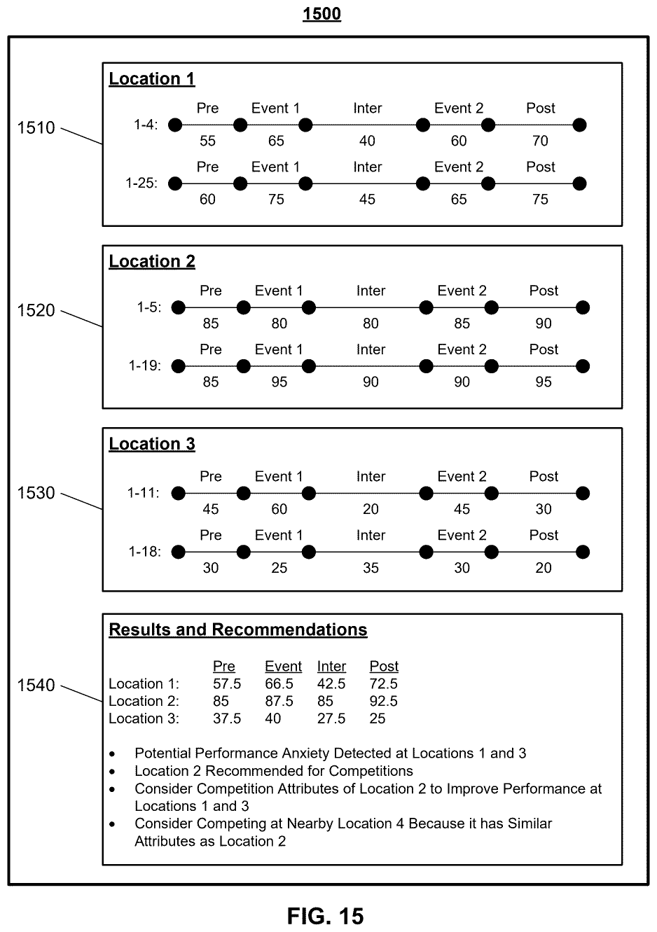

FIG. 15 illustrates an example user interface 1500 related to computed location-based user-specific performance and mindfulness scores with associated recommendations generated by the system 200. The user interface 1500 is a user-specific interface associated with competition-based performance and mindfulness activities of a particular user at different locations. The system 200 generates the user interface 1500 using user-specific sensor data associated with the particular user during competition events, as well as location-specific sensor data associated with the locations where the particular user engages in competitions.

The user interface 1500 shows different competition events tracked for a particular user at three different locations. In this example, the system 200 determined that the user engaged in competition activities (e.g., swimming competitions) at each of the three locations. As part of the detection, the system 200 determined performance-based mindfulness scores for the particular user during each of the competition events. In this example, the system 200 detected the particular user as engaging in two competition events per day on each occasion, although more or fewer competition events per day could be tracked by the system 200.

The user interface 1500 includes a first section 1510 that shows competition events tracked at a first location. As shown, the user engaged in two competition events on January 4 at the first location. The system 200 generated a pre-event mindfulness score of 55 that reflects the particular user's mindfulness prior to competition, an event 1 mindfulness score of 65 that reflects the particular user's mindfulness during the first event, an interevent mindfulness score of 40 that reflects the particular user's mindfulness between the two events, an event 2 mindfulness score of 60 that reflects the particular user's mindfulness during the second event, and a post-event mindfulness score of 70 that reflects the particular user's mindfulness after the second event. As shown in the first section 1510, the system 200 detected a similar pattern of performance-based mindfulness scores for the same two events that occurred on January 25.

The user interface 1500 also include a second section 1520 and a third section 1530. The second section 1520 and the third section 1530 are both similar to the first section 1510, but associated with a second location and a third location, respectively. As shown, the system 200 detected the same two event competition pattern for the particular user at the second and third locations and computed the same types of performance-based mindfulness scores related to pre-event mindfulness, during event mindfulness, interevent, mindfulness and post-event mindfulness.

The user interface 1500 further includes a results and recommendations section 1540. As shown in the section 1540, the system 200 computed overall performance-based mindfulness scores for each of the first, second, and third locations. As shown, the system 200 presents pre-event performance-based mindfulness scores, during event performance-based mindfulness scores, interevent performance-based mindfulness scores, and post-event performance-based mindfulness scores. The system 200 may compute these scores as averages of the specific event scores shown in the first, second, and third sections 1510 to 1530.

The results in the recommendation section 1540 also include information inferred by the system 200 as well as recommendations to improve performance-based mindfulness. As shown by the first recommendation, the system 200 detected potential performance anxiety at the first and third locations. The system 200 made this detection by comparing the performance-based mindfulness scores computed at the first and third locations relative to the performance-based mindfulness scores computed at the second location, where the scores were significantly higher. The system 200 may account for both the pre-event performance-based mindfulness scores and the interevent performance-based mindfulness scores in determining whether the particular user is suffering from performance anxiety at any of the locations. In some examples, the system 200 may weight pre-event performance-based mindfulness scores higher than interevent performance-based mindfulness scores in determining whether the particular user is suffering from performance anxiety.

The second recommendation in the section 1540 recommends that the particular user engage in competitions at the second location if possible. Because the system 200 detected that the particular user has higher performance-based mindfulness scores at the second location as compared to the first and third locations, the system 200 recommends that the particular user try to compete at the second location when possible and recommends that to the particular user.

The third recommendation in the section 1540 recommends that the particular user consider competition attributes of the second location to improve performance at the first and third locations. In this example, the system 200 determines that attributes of the venues located at the first and third locations may be causing the particular user more anxiety than the attributes at the second location. Accordingly, the system 200 recommends that the user attempt to adjust the competition attributes at the first and third location to match those of the second location. These adjustments may be recommended or automatically adjusted by the system 200, and may include lighting considerations, music considerations, noise considerations, considerations of the people that the user associates with at each of the locations, the environment in which the user must wait for events at each of the locations, etc.

The final recommendation in the section 1540 recommends that the particular user consider competing at nearby location 4 because it has similar attributes to the second location. The system 200 automatically compares the attributes of the second location to attributes of other locations where the user has not competed, but that are within a threshold distance of the first, second, or third locations. The system 200 automatically chose to compare the attributes of the second location to the attributes of other locations because the particular user had relatively high performance-based mindfulness scores at the second location and the particular user might benefit from competing at locations that have similar attributes.

The described systems, methods, and techniques may be implemented in digital electronic circuitry, computer hardware, firmware, software, or in combinations of these elements. Apparatus implementing these techniques may include appropriate input and output devices, a computer processor, and a computer program product tangibly embodied in a machine-readable storage device for execution by a programmable processor. A process implementing these techniques may be performed by a programmable processor executing a program of instructions to perform desired functions by operating on input data and generating appropriate output. The techniques may be implemented in one or more computer programs that are executable on a programmable system including at least one programmable processor coupled to receive data and instructions from, and to transmit data and instructions to, a data storage system, at least one input device, and at least one output device. Each computer program may be implemented in a high-level procedural or object-oriented programming language, or in assembly or machine language if desired; and in any case, the language may be a compiled or interpreted language. Suitable processors include, by way of example, both general and special purpose microprocessors. Generally, a processor will receive instructions and data from a read-only memory and/or a random access memory. Storage devices suitable for tangibly embodying computer program instructions and data include all forms of non-volatile memory, including by way of example semiconductor memory devices, such as Erasable Programmable Read-Only Memory (EPROM), Electrically Erasable Programmable Read-Only Memory (EEPROM), and flash memory devices; magnetic disks such as internal hard disks and removable disks; magneto-optical disks; and Compact Disc Read-Only Memory (CD-ROM). Any of the foregoing may be supplemented by, or incorporated in, specially-designed ASICs (application-specific integrated circuits).

It will be understood that various modifications may be made. For example, other useful implementations could be achieved if steps of the disclosed techniques were performed in a different order and/or if components in the disclosed systems were combined in a different manner and/or replaced or supplemented by other components. Accordingly, other implementations are within the scope of the disclosure.

What is claimed is:

1. An electronic system configured to perform location-based cardiovascular monitoring for performance and mindfulness sensing, the electronic system comprising:
a plurality of user devices that each include or are connected to (1) at least one user-specific biological sensor configured to sense biological characteristics of a specific user, (2) at least one user-specific activity sensor configured to sense activity characteristics of the specific user, (3) at least one user-specific environmental sensor configured to sense environmental characteristics of an area where the specific user is located when the biological and activity characteristics of the specific user are sensed, and (4) at least one user-specific location sensor configured to sense a location of the specific user when the biological and activity characteristics of the specific user are sensed;
a plurality of location-specific devices that each include or are connected to at least one location-specific environmental sensor configured to sense environmental characteristics at a specific location, the location-specific devices and corresponding location-specific environmental sensors being different from the user devices and corresponding user-specific environmental sensors;
at least one database configured to store (1) user-specific biological data sensed by user-specific biological sensors, (2) user-specific activity data sensed by user-specific activity sensors, (3) user-specific environmental data sensed by user-specific environmental sensors, (4) user-specific location data sensed by user-specific location sensors, and (5) location-specific environmental data sensed by location-specific environmental sensors; and
at least one monitoring server configured to communicate with the plurality of user devices, the plurality of location-based devices, and the at least one database, the at least one monitoring server comprising one or more processors and one or more computer-readable media storing instructions that, when executed, cause at least one of the one or more processors to perform operations comprising:
receiving, over time and from the plurality of user devices, (1) user-specific biological data sensed by user-specific biological sensors configured to sense biological characteristics of specific users, (2) user-specific activity data sensed by user-specific activity sensors configured to sense activity characteristics of specific users, (3) user-specific environmental data sensed by user-specific environmental sensors configured to sense environmental characteristics of areas where specific users are located when biological and activity characteristics are sensed, and (4) user-specific location data sensed by user-specific location sensors configured to sense locations of specific users when biological and activity characteristics are sensed;
receiving, over time and from the plurality of location-specific devices, location-specific environmental data sensed by location-specific environmental sensors configured to sense environmental characteristics at specific locations;
storing, in the at least one database, the received (1) user-specific biological data, (2) user-specific activity data, (3) user-specific environmental data, (4) user-specific location data, and (5) location-specific environmental data in association with timing data indicating a time of sensing;
based on the stored (1) user-specific biological data, (2) user-specific activity data, (3) user-specific environmental data, (4) user-specific location data, and (5) location-specific environmental data:
computing location-based user-specific performance scores that each measure health and performance levels for a specific user at a specific location, the location-based user-specific performance scores comprising at least:
a first location-based user-specific performance score that measures health and performance levels for a first user at a first location,
a second location-based user-specific performance score that measures health and performance levels for the first user at a second location, the second location being different from the first location,
a third location-based user-specific performance score that measures health and performance levels for a second user at the first location, the second user being different from the first user, and
a fourth location-based user-specific performance score that measures health and performance levels for the second user at the second location;
computing location-based user-specific mindfulness scores that each measure mindfulness levels for a specific user at a specific location, the location-based user-specific mindfulness scores comprising at least:
a first location-based user-specific mindfulness score that measures mindfulness levels for the first user at the first location,
a second location-based user-specific mindfulness score that measures mindfulness levels for the first user at the second location,
a third location-based user-specific mindfulness score that measures mindfulness levels for the second user at the first location, and
a fourth location-based user-specific mindfulness score that measures mindfulness levels for the second user at the second location;
computing location-specific performance scores that each measure health and performance levels associated with a specific location, the location-specific performance scores comprising at least:
a first location-specific performance score that measures health and performance levels associated with the first location, the first location-specific performance score accounting for all biological data, activity data, and environmental data sensed at the first location, and
a second location-specific performance score that measures health and performance levels associated with the second location, the second location-specific performance score accounting for all biological data, activity data, and environmental data sensed at the second location; and
computing location-specific mindfulness scores that each measure mindfulness levels associated with a specific location, the location-specific mindfulness scores comprising at least:
a first location-specific mindfulness score that measures mindfulness levels associated with the first location, the first location-specific mindfulness score accounting for all biological data, activity data, and environmental data sensed at the first location, and
a second location-specific mindfulness score that measures mindfulness levels associated with the second location, the second location-specific mindfulness score accounting for all biological data, activity data, and environmental data sensed at the second location; and
performing at least one operation based on the computed location-based user-specific performance scores, the computed location-based user-specific mindfulness scores, the computed location-specific performance scores, and the computed location-specific mindfulness scores.

2. The system of claim 1, wherein computing the first location-based user-specific performance score, the second location-based user-specific performance score, the first location-based user-specific mindfulness score, and the second location-based user-specific mindfulness score comprises:
filtering the stored (1) user-specific biological data, (2) user-specific activity data, (3) user-specific environmental data, and (4) user-specific location data for the first user;
clustering the filtered (1) user-specific biological data, (2) user-specific activity data, and (3) user-specific environmental data for the first user by the (4) user-specific location data for the first user to identify clusters of biological data, activity data, and environmental data for the first user;
selecting, from among the clusters of biological data, activity data, and environmental data for the first user, location clusters of statistically significant data for the first user;
disregarding, from among the clusters of biological data, activity data, and environmental data for the first user, insignificant clusters for the first user while maintaining data from the insignificant clusters for further monitoring;
determining whether location-specific environmental data is available for each of the selected location clusters of statistically significant data for the first user;
based on a determination that location-specific environmental data exists for each of the selected location clusters of statistically significant data for the first user, add location-specific environmental data to each corresponding location cluster of statistically significant data for the first user to create sets of location-specific data for the first user, the sets of location-specific data for the first user comprising a first set of location-specific data for the first user at the first location and a second set of location-specific data for the first user at the second location;
processing the first set of location-specific data for the first user at the first location to compute the first location-based user-specific performance score and the first location-based user-specific mindfulness score; and
processing the second set of location-specific data for the first user at the second location to compute the second location-based user-specific performance score and the second location-based user-specific mindfulness score.

3. The system of claim 2:
wherein filtering the stored (1) user-specific biological data, (2) user-specific activity data, (3) user-specific environmental data, and (4) user-specific location data for the first user comprises filtering the stored (1) user-specific biological data, (2) user-specific activity data, (3) user-specific environmental data, and (4) user-specific location data for the first user into a first subset of performance-related data, a second subset of mindfulness-related data, and a third subset of unrelated data; and
wherein clustering the filtered (1) user-specific biological data, (2) user-specific activity data, and (3) user-specific environmental data for the first user by the (4) user-specific location data for the first user to identify clusters of biological data, activity data, and environmental data for the first user comprises:
disregarding the third subset of unrelated data,
identifying clusters of performance-related data using the first subset of performance-related data, the clusters of performance-related data being used to compute the first location-based user-specific performance score and the second location-based user-specific performance score, and
identifying clusters of mindfulness-related data using the second subset of performance-related data, the clusters of mindfulness-related data being used to compute the first location-based user-specific mindfulness score and the second location-based user-specific mindfulness score.

4. The system of claim 3, wherein filtering the stored (1) user-specific biological data, (2) user-specific activity data, (3) user-specific environmental data, and (4) user-specific location data for the first user into a first subset of performance-related data, a second subset of mindfulness-related data, and a third subset of unrelated data comprises:
arranging, into the first subset of performance-related data, user-specific biological data where a heart rate measurement is above a performance threshold value and user-specific activity data where an activity level is above a performance threshold level of activity and an activity pattern matches an exercise activity pattern;
arranging, into the second subset of mindfulness-related data, user-specific biological data where a heart rate measurement is below a mindfulness threshold value and user-specific activity data where an activity level is below a mindfulness threshold level of activity and an activity pattern matches a mindfulness activity pattern; and
arranging all remaining data into the third subset of unrelated data.

5. The system of claim 1, wherein computing the first location-specific performance score, the second location-specific performance score, the first location-specific mindfulness score, and the second location-specific mindfulness score comprises:
clustering the stored (1) user-specific biological data, (2) user-specific activity data, and (3) user-specific environmental data by the (4) user-specific location data to identify clusters of biological data, activity data, and environmental data;
selecting, from among the clusters of biological data, activity data, and environmental data, location clusters of statistically significant data;
disregarding, from among the clusters of biological data, activity data, and environmental data, insignificant clusters while maintaining data from the insignificant clusters for further monitoring;
accessing, from the at least one database, known location data that indicates known locations for performance and mindfulness;
comparing locations for each of the selected location clusters of biological data, activity data, and environmental data with known locations from the accessed known location data;
based on the comparison of the locations for each of the selected location clusters of biological data, activity data, and environmental data with known locations from the accessed known location data, performing location verification and location inference processing to identify verified locations for performance and mindfulness and inferred locations for performance and mindfulness, wherein performing location verification processing comprises identifying each of the first location and the second location as verified locations;
based on identification of each of the first location and the second location as verified locations:
accessing, from the at least one database, first location-specific environmental data for the first location and second location-specific environmental data for the second location,
adding the first location-specific environmental data for the first location to a first location cluster of biological data, activity data, and environmental data that includes biological data, activity data, and environmental data received from all users at the first location to create a first set of location-specific data for the first location, and
adding the second location-specific environmental data for the second location to a second location cluster of biological data, activity data, and environmental data that includes biological data, activity data, and environmental data received from all users at the second location to create a second set of location-specific data for the second location;
processing the first set of location-specific data for the first location to compute the first location-specific performance score and the first location-specific mindfulness score; and
processing the second set of location-specific data for the second location to compute the second location-specific performance score and the second location-specific mindfulness score.

6. The system of claim 5, wherein computing location-specific performance scores and computing location-specific mindfulness scores further comprises:
selecting, from among the inferred locations for performance and mindfulness identified through location inference processing, a first inferred location for performance;
selecting, from among the inferred locations for performance and mindfulness identified through location inference processing, a second inferred location for mindfulness;
computing a third location-specific performance score that measures health and performance levels associated with the first inferred location for performance, the third location-specific performance score accounting for all biological data, activity data, and environmental data sensed at the first inferred location for performance;
computing a third location-specific mindfulness score that measures mindfulness levels associated with the second inferred location for mindfulness, the third location-specific mindfulness score accounting for all biological data, activity data, and environmental data sensed at the second inferred location for mindfulness; and
causing display of a map view that presents the first location, the second location, the first inferred location, and the second inferred location in relative geographic positions on a map with indications that the first and second locations are verified locations and the first and second inferred locations are inferred locations, the map view presenting the first location-specific performance score and the first location-specific mindfulness score in association with the first location, the second location-specific performance score and the second location-specific mindfulness score in association with the second location, the third location-specific performance score in association with the first inferred location, and the third location-specific mindfulness score in association with the second inferred location.

7. The system of claim 5, wherein performing location verification processing comprises:
based on the comparison of the locations for each of the selected location clusters of biological data, activity data, and environmental data with known locations from the accessed known location data, determining whether the locations for each of the selected location clusters match the known locations from the accessed known location data to identify a set of matching location clusters and a set of non-matching location clusters;
disregarding the set of non-matching location clusters as unverified locations while retaining the set of non-matching location clusters for continued monitoring;
identifying, from the accessed known location data, types of locations for matching location clusters in the set of matching location clusters;
determining, for the set of matching location clusters, types of activity performed at matching location clusters in the set of matching location clusters based on an analysis of biological data, activity data, and environmental data included in each matching location cluster in the set of matching location clusters;
comparing the types of locations for matching location clusters in the set of matching location clusters with the types of activity performed at matching location clusters in the set of matching location clusters;
based on the comparison of the types of locations for matching location clusters in the set of matching location clusters with the types of activity performed at matching location clusters in the set of matching location clusters, identifying a first subset of the set of matching location clusters where the type of location matches the type of activity and a second subset of the set of matching location clusters where the type of location does not match the type of activity;
disregarding the second subset of the set of matching location clusters as unverified locations while retaining the second subset of the set of matching location clusters for continued monitoring; and
verifying the first subset of the set of matching location clusters as verified locations.

8. The system of claim 7, wherein performing location verification processing further comprises:
causing display of the set of non-matching location clusters with user interface controls that enable a user to designate one or more of the non-matching locations in the set of non-matching location clusters as verified locations;
determining, based on user input received through the user interface controls, that the user has designated the one or more of the non-matching locations in the set of non-matching location clusters as verified locations;
verifying the one or more of the non-matching locations as verified locations; and
updating the known location data to include the one or more of the non-matching locations verified based on the user input.

9. The system of claim 5, wherein performing location inference processing comprises:
based on the comparison of the locations for each of the selected location clusters of biological data, activity data, and environmental data with known locations from the accessed known location data, determining whether the locations for each of the selected location clusters match the known locations from the accessed known location data to identify a set of non-matching location clusters;
determining, for the set of non-matching location clusters, types of activity performed at non-matching location clusters in the set of non-matching location clusters based on an analysis of biological data, activity data, and environmental data included in each non-matching location cluster in the set of non-matching location clusters;
determining whether the types of activity performed at non-matching location clusters in the set of non-matching location clusters match performance or mindfulness activities to identify a first subset of non-matching location clusters inferred as matching performance activities, a second subset of non-matching location clusters inferred as matching mindfulness activities, a third subset of non-matching location clusters inferred as matching performance and mindfulness activities, and a fourth subset of non-matching location clusters where the types of activity did not match performance or mindfulness activities;
disregarding the fourth subset of non-matching location clusters as inconclusive locations while retaining the fourth subset of non-matching location clusters for continued monitoring;
designating the first subset of non-matching location clusters as inferred performance locations;
designating the second subset of non-matching location clusters as inferred mindfulness locations; and
designating the third subset of non-matching location clusters as inferred performance and mindfulness locations.

10. The system of claim 9:
wherein the inferred performance locations includes a first inferred location for performance;
wherein the inferred mindfulness locations includes a first inferred location for mindfulness;
wherein the inferred performance and mindfulness locations includes a first inferred location for performance and mindfulness;
wherein computing location-specific performance scores and computing location-specific mindfulness scores further comprises:
computing a third location-specific performance score that measures health and performance levels associated with the first inferred location for performance, the third location-specific performance score accounting for all biological data, activity data, and environmental data sensed at the first inferred location for performance;
computing a third location-specific mindfulness score that measures mindfulness levels associated with the first inferred location for mindfulness, the third location-specific mindfulness score accounting for all biological data, activity data, and environmental data sensed at the first inferred location for mindfulness;
computing a fourth location-specific performance score that measures health and performance levels associated with the first inferred location for performance and mindfulness, the fourth location-specific performance score accounting for all biological data, activity data, and environmental data sensed at the first inferred location for performance and mindfulness;
computing a fourth location-specific mindfulness score that measures mindfulness levels associated with the first inferred location for performance and mindfulness, the fourth location-specific mindfulness score accounting for all biological data, activity data, and environmental data sensed at the first inferred location for performance and mindfulness; and
wherein performing the at least one operation based on the computed location-based user-specific performance scores, the computed location-based user-specific mindfulness scores, the computed location-specific performance scores, and the computed location-specific mindfulness scores comprises causing display of a user interface that (1) suggests the first inferred location for performance and the first inferred location for performance and mindfulness as inferred locations for performance activities, (2) suggests the first inferred location for mindfulness and the first inferred location for performance and mindfulness as inferred locations for mindfulness activities, (3) presents the third location-specific performance score in association with the first inferred location for performance as part of the suggestion of inferred locations for performance activities, (4) presents the fourth location-specific performance score in association with the first inferred location for performance and mindfulness as part of the suggestion of inferred locations for performance activities, (5) presents the third location-specific mindfulness score in association with the first inferred location for mindfulness as part of the suggestion of inferred locations for mindfulness activities, and (6) presents the fourth location-specific mindfulness score in association with the first inferred location for performance and mindfulness as part of the suggestion of inferred locations for mindfulness activities.

11. The system of claim 9, wherein performing location inference processing further comprises updating location inference processing for the fourth subset of non-matching location clusters based on additional data received from the plurality of user devices by:
determining that a location within the fourth subset of non-matching location clusters has types of activity that match performance activities during a subset of a year based on the additional data received from the plurality of user devices;
based on the determination that the location within the fourth subset of non-matching location clusters has types of activity that match performance activities during the subset of the year, designating the location within the fourth subset of non-matching location clusters as an inferred seasonal performance location; and
processing the inferred seasonal performance location based on data only from the subset of the year.

12. The system of claim 5, wherein clustering the stored (1) user-specific biological data, (2) user-specific activity data, and (3) user-specific environmental data by the (4) user-specific location data to identify clusters of biological data, activity data, and environmental data comprises:
filtering the stored (1) user-specific biological data, (2) user-specific activity data, (3) user-specific environmental data, and (4) user-specific location data into a first subset of performance-related data, a second subset of mindfulness-related data, and a third subset of unrelated data; and
disregarding the third subset of unrelated data,
identifying clusters of performance-related data by location using the first subset of performance-related data, the clusters of performance-related data being used to compute the first location-specific performance score and the second location-specific performance score, and
identifying clusters of mindfulness-related data by location using the second subset of performance-related data, the clusters of mindfulness-related data being used to compute the first location-specific mindfulness score and the second location-specific mindfulness score.

13. The system of claim 12, wherein filtering the stored (1) user-specific biological data, (2) user-specific activity data, (3) user-specific environmental data, and (4) user-specific location data into the first subset of performance-related data, the second subset of mindfulness-related data, and the third subset of unrelated data comprises:
arranging, into the first subset of performance-related data, user-specific biological data where a heart rate measurement is above a performance threshold value and user-specific activity data where an activity level is above a performance threshold level of activity and an activity pattern matches an exercise activity pattern;
arranging, into the second subset of mindfulness-related data, user-specific biological data where a heart rate measurement is below a mindfulness threshold value and user-specific activity data where an activity level is below a mindfulness threshold level of activity and an activity pattern matches a mindfulness activity pattern; and
arranging all remaining data into the third subset of unrelated data.

14. The system of claim 1, wherein computing location-based user-specific performance scores that each measure health and performance levels for a specific user at a specific location comprises:
processing the stored user-specific activity data to identify performance activities performed by a specific user at a specific location;
identifying, within the stored user-specific biological data and the stored user-specific environmental data, a first subset of user-specific biological data and a second subset of user-specific environmental data that are associated with the identified performance activities;
analyzing performance levels for the identified performance activities with the first subset of user-specific biological data in view of the second subset of user-specific environmental data;
based on the analysis of the performance levels for the identified performance activities with the first subset of user-specific biological data in view of the second subset of user-specific environmental data, determining a series of performance scores corresponding to the identified performance activities; and
computing an overall performance score for the specific user at the specific location based on the series of performance scores corresponding to the identified performance activities.

15. The system of claim 1, wherein computing location-based user-specific mindfulness scores that each measure mindfulness levels for a specific user at a specific location comprises:
processing the stored user-specific activity data to identify mindfulness activities performed by a specific user at a specific location;
identifying, within the stored user-specific biological data and the stored user-specific environmental data, a first subset of user-specific biological data and a second subset of user-specific environmental data that are associated with the identified mindfulness activities;
analyzing mindfulness levels for the identified mindfulness activities with the first subset of user-specific biological data in view of the second subset of user-specific environmental data;
based on the analysis of the mindfulness levels for the identified mindfulness activities with the first subset of user-specific biological data in view of the second subset of user-specific environmental data, determining a series of mindfulness scores corresponding to the identified mindfulness activities; and
computing an overall mindfulness score for the specific user at the specific location based on the series of mindfulness scores corresponding to the identified mindfulness activities.

16. The system of claim 1, wherein computing location-specific performance scores that each measure health and performance levels associated with a specific location comprises:
processing the stored user-specific activity data to identify performance activities performed by all users at a specific location;
identifying, within the stored user-specific biological data and the stored user-specific environmental data, a first subset of user-specific biological data and a second subset of user-specific environmental data that are associated with the identified performance activities performed by all users at the specific location;
analyzing performance levels for the identified performance activities performed by all users at the specific location with the first subset of user-specific biological data in view of the second subset of user-specific environmental data;
based on the analysis of the performance levels for the identified performance activities with the first subset of user-specific biological data in view of the second subset of user-specific environmental data, determining a series of performance scores corresponding to the identified performance activities performed by all users at the specific location; and
computing an overall performance score for the specific location based on the series of performance scores corresponding to the identified performance activities performed by all users at the specific location.

17. The system of claim 1, wherein computing location-specific mindfulness scores that each measure mindfulness levels associated with a specific location comprises:
processing the stored user-specific activity data to identify mindfulness activities performed by all users at a specific location;
identifying, within the stored user-specific biological data and the stored user-specific environmental data, a first subset of user-specific biological data and a second subset of user-specific environmental data that are associated with the identified mindfulness activities performed by all users at the specific location;
analyzing mindfulness levels for the identified mindfulness activities performed by all users at the specific location with the first subset of user-specific biological data in view of the second subset of user-specific environmental data;
based on the analysis of the mindfulness levels for the identified mindfulness activities with the first subset of user-specific biological data in view of the second subset of user-specific environmental data, determining a series of mindfulness scores corresponding to the identified mindfulness activities performed by all users at the specific location; and
computing an overall mindfulness score for the specific location based on the series of mindfulness scores corresponding to the identified mindfulness activities performed by all users at the specific location.

18. The system of claim 1, wherein computing location-based user-specific mindfulness scores that each measure mindfulness levels for a specific user at a specific location comprises computing performance-based, location-based user-specific mindfulness scores that each measure mindfulness levels for a specific user at a specific location related to performing a performance activity by:
processing the stored user-specific activity data to identify performance activities performed by a specific user at a specific location;
identifying, within the stored user-specific biological data and the stored user-specific activity data, a first subset of user-specific biological and activity data that precedes the identified performance activities, a second subset of user-specific biological and activity data during the identified performance activities, and a third subset of user-specific biological and activity data that follows the identified performance activities;
analyzing the first subset of user-specific biological and activity data that precedes the identified performance activities to assess mindfulness of the specific user prior to the identified performance activities;
analyzing the second subset of user-specific biological and activity data during the identified performance activities to assess mindfulness of the specific user during the identified performance activities;
analyzing the third subset of user-specific biological and activity data that follows the identified performance activities to assess mindfulness of the specific user following the identified performance activities; and
computing performance-based, location-based user-specific mindfulness scores based on the analysis of the first, second, and third subsets of user-specific biological and activity data.

19. The system of claim 18:
wherein processing the stored user-specific activity data to identify performance activities performed by the specific user at the specific location comprises identifying a first set of training-based performance activities and a second set of competition-based performance activities;
wherein computing performance-based, location-based user-specific mindfulness scores based on the analysis of the first, second, and third subsets of user-specific biological and activity data comprises:
computing training performance-based, location-based user-specific mindfulness scores based on the analysis of the first, second, and third subsets of user-specific biological and activity data for the first set of training-based performance activities, and
computing competition performance-based, location-based user-specific mindfulness scores based on the analysis of the first, second, and third subsets of user-specific biological and activity data for the second set of competition-based performance activities;
wherein performing at least one operation based on the computed location-based user-specific performance scores, the computed location-based user-specific mindfulness scores, the computed location-specific performance scores, and the computed location-specific mindfulness scores comprises:
comparing the training performance-based, location-based user-specific mindfulness scores to the competition performance-based, location-based user-specific mindfulness scores,
based on the comparison of the training performance-based, location-based user-specific mindfulness scores to the competition performance-based, location-based user-specific mindfulness scores, determining whether the training performance-based, location-based user-specific mindfulness scores are similar to the competition performance-based, location-based user-specific mindfulness scores,
based on a determination that the training performance-based, location-based user-specific mindfulness scores are dissimilar to the competition performance-based, location-based user-specific mindfulness scores, determining that the specific user is suspected of experiencing competition anxiety,
based on the determination that the specific user is suspected of experiencing competition anxiety:
identifying a pre-competition subset of user-specific biological and activity data that precedes identified competition-based performance activities,
identifying an inter-competition subset of user-specific biological and activity data that falls between identified competition-based performance activities within a threshold period of time,
determining a pre-competition mindfulness score based on the pre-competition subset of user-specific biological and activity data,
determining an inter-competition mindfulness score based on the inter-competition subset of user-specific biological and activity data,
analyzing the competition performance-based, location-based user-specific mindfulness scores for variation across different locations, and
based on the analysis of the competition performance-based, location-based user-specific mindfulness scores for variation across different locations, identifying at least one location with improved competition mindfulness for the specific user; and
wherein performing the at least one operation based on the computed location-based user-specific performance scores, the computed location-based user-specific mindfulness scores, the computed location-specific performance scores, and the computed location-specific mindfulness scores comprises:
causing display of a user interface that presents the pre-competition mindfulness score and the inter-competition mindfulness score to the specific user,
determining attributes of the identified at least one location with improved competition mindfulness for the specific user, and
causing display of a user interface with a suggestion to compete at the identified at least one location with improved competition mindfulness for the specific user and a suggestion to compete at other locations with attributes similar to the identified at least one location with improved competition mindfulness for the specific user.

20. The system of claim 1, wherein performing the at least one operation based on the computed location-based user-specific performance scores, the computed location-based user-specific mindfulness scores, the computed location-specific performance scores, and the computed location-specific mindfulness scores comprises:
identifying recommended performance locations for a specific user based on the computed location-based user-specific performance scores for the specific user, the recommended performance locations for the specific user being performance locations where the specific user's location-based user-specific performance score exceeds a threshold performance score;
identifying recommended mindfulness locations for the specific user based on the computed location-based user-specific mindfulness scores for the specific user, the recommended mindfulness locations for the specific user being mindfulness locations where the specific user's location-based user-specific mindfulness score exceeds a threshold mindfulness score;
identifying suggested performance locations for the specific user based on the computed location-specific performance scores, the suggested performance locations for the specific user being performance locations where the specific user does not have a location-based user-specific performance score and the computed location-specific performance score exceeds a threshold performance score;
identifying suggested mindfulness locations for the specific user based on the computed location-specific mindfulness scores, the suggested mindfulness locations for the specific user being mindfulness locations where the specific user does not have a location-based user-specific mindfulness score and the computed location-specific mindfulness score exceeds a threshold mindfulness score;
identifying a low-performing performance location based on the computed location-specific performance scores, the low-performing performance location being a performance location where the computed location-specific performance score falls below the threshold performance score;
identifying a low-performing mindfulness location based on the computed location-specific mindfulness scores, the low-performing mindfulness location being a mindfulness location where the computed location-specific mindfulness score falls below the threshold mindfulness score;
causing display of a user interface that presents, to the specific user, the recommended performance locations for the specific user, the recommended mindfulness locations for the specific user, the suggested performance locations for the specific user, the suggested mindfulness locations for the specific user, and indications to avoid the low-performing performance location and the low-performing mindfulness location;
determining attributes of the suggested performance locations that differ from attributes of the low-performing performance location;
causing display of a user interface that presents, to a first owner of the low-performing performance location, a suggestion for improving the low-performing performance location that identifies the attributes of the suggested performance locations that differ from attributes of the low-performing performance location;
determining attributes of the suggested mindfulness locations that differ from attributes of the low-performing mindfulness location; and
causing display of a user interface that presents, to a second owner of the low-performing mindfulness location, a suggestion for improving the low-performing mindfulness location that identifies the attributes of the suggested mindfulness locations that differ from attributes of the low-performing mindfulness location.
